SEQUENCE LISTING (9 sequences)

SEQ ID NO: 1 - DNA, 2147 bp, Artificial Sequence
  Synthetic sequence
ctttggggagtcctaagagggcagctggcaatggacacctagcagtccctttgagacttatttcagatggagctgtagaaagatgccatggctcacagtgcctccctgggaagggggcagagggctgcccagtgaggcctcttgcgagcaggaaatcaccagagacaaggaaagaccagaccccaggatgacctcagttaggccttgcccgactgtcctcagagtcccattctctgtgtcctggttcttttagaagatcatggacctccaggtcatttcgtaaccggaatctgcctgcggggggttttgacaagctatggtatagtgtatgtgggggtactgacgaattggaagatcatggagaccccttctcctcctccatcattggtctgccacatccctcccaggcgactcacagcagagagaccttggatgtatgtagggtgctttaaaactccagctgagttacagtctctcctttctgttttcaccttaaccttccagggatgcaaacccacgacaggtttagcagcagagtggaggctggccatgaatctcagagaaagtgctcactggaaaggctggtttagcccaggcctgatgtggaggcactgagctggacgttctagcggggttgacacccaacagtttacatagggggaggccacccctcctgagcagtctcggtgacttgaagaggaagccgcttcttctgtaccaacacagaagctccagcgaacccccagaatgctggcagtgtgggtgctatgtaaaagtatttacatagctttgtagagtgagccaagcccagtctgtttgggatgactcttcacagtgcctcgaatctgtcacacgtcttagtaagcagagtcacagagtttctgtcacatcatcctcctgcctacagggaagtaggccatgtccctgccccctactctgagcccagctgtgggagccagccctgcccaatgggctctctctgattggcttctcactcacttctaaactccagtgagcaacttctctcggctcgttcaattggcgtgaaggtctgtgtcttgcagagaaggttcttcacaactgggataaaggtctcgctgctcaagtgtagcccagtagaactgccaagccccttcccctcctctccctagactcttggatgcaagaagaatccaggcagctccaagggtgattgtgtccaacctagaatgtcttgaaaaagacattaaggggactagagaagacaggggatccaacggttctctgcagcccagcctgactgacatgtaactcttctggttctcaccagccagctggacctgcttagtattctttctgcctcagtttcccagcctgtacccagggctgtcatagttccatttcaggcagtagtaatgaatgagctgacataaaacatttagagcaggggtcagtatgtatatagagtgattattctatatcaggcattgcctcctcggaatgaagcttacaatcacccctccctctgcagttcatcttggggtggccagaggatccagcagacacctagtggggtaacacaccccagccaactcggctgttgcagactttgtctagaagtttcacgtctcagagctgaattcccttctcatgacctttggccgtgggagtgacacctcacagctgtggtgttttgacaaccagcagccactggcacacaaaatgtgcagccagcagcatatgaagtccaagaggcgtcccggccagccctgtccttgacccccacctgacaattaaggcaagagcctatagtttgcatcagcaacagtcacggtcaaagtttagtcaatcaaacgttgtgtaaggactcaactatggctgacacgggggcctgaggcctcccaacattcattaacaacagcaagttcaatcattatctccccaaagtttattgtgttaggtcagttccaaaccgtgctgaccatggctatgatccaaaggccggccccttacgtcagaggcgagcctccaggtccagctgaggggcagggctgtcctcccttctgtatactatttaaagcgaggagggctagctaccaagcacggttggccttccctctgggaacacacccttggccaacaggggaaatccggcgagacgctctgag

SEQ ID NO: 2 - DNA, 127 bp, Artificial Sequence
  synthetic sequence
atcctgcgagaaggaggtgcgtcctgctgcctgccccggcactctggctccccagctcaaggttcaggccttgccccaggccgggcctctgggtacctgaggtcttctcccgctctgtgcccttctc

SEQ ID NO: 3 - DNA, 1431 bp, Homo sapiens
atggcgtcacgcatagggttgcgcatgcagctcatgcgggagcaggcgcagcaggaggagcagcgggagcgcatgcagcaacaggctgtcatgcattacatgcagcagcagcagcagcagcaacagcagcagctcggagggccgcccaccccggccatcaatacccccgtccacttccagtcgccaccacctgtgcctggggaggtgttgaaggtgcagtcctacctggagaatcccacatcctaccatctgcagcagtcgcagcatcagaaggtgcgggagtacctgtccgagacctatgggaacaagtttgctgcccacatcagcccagcccagggctctccgaaacccccaccagccgcctccccaggggtgcgagctggacacgtgctgtcctcctccgctggcaacagtgctcccaatagccccatggccatgctgcacattggctccaaccctgagagggagttggatgatgtcattgacaacattatgcgtctggacgatgtccttggctacatcaatcctgaaatgcagatgcccaacacgctacccctgtccagcagccacctgaatgtgtacagcagcgacccccaggtcacagcctccctggtgggcgtcaccagcagctcctgccctgcggacctgacccagaagcgagagctcacagatgctgagagcagggccctggccaaggagcggcagaagaaagacaatcacaacttaattgaaaggagacgaaggttcaacatcaatgaccgcatcaaggagttgggaatgctgatccccaaggccaatgacctggacgtgcgctggaacaagggcaccatcctcaaggcctctgtggattacatccggaggatgcagaaggacctgcaaaagtccagggagctggagaaccactctcgccgcctggagatgaccaacaagcagctctggctccgtatccaggagctggagatgcaggctcgagtgcacggcctccctaccacctccccgtccggcatgaacatggctgagctggcccagcaggtggtgaagcaggagctgcctagcgaagagggcccaggggaggccctgatgctgggggctgaggtccctgaccctgagccactgccagctctgcccccgcaagccccgctgcccctgcccacccagccaccatccccattccatcacctggacttcagccacagcctgagctttgggggcagggaggacgagggtcccccgggctaccccgaacccctggcgccggggcatggctccccattccccagcctgtccaagaaggatctggacctcatgctcctggacgactcactgctaccgctggcctctgatccacttctgtccaccatgtcccccgaggcctccaaggccagcagccgccggagcagcttcagcatggaggagggcgatgtgctgtga

SEQ ID NO: 4 - protein, 476 aa, Homo sapiens
MASRIGLRMQLMREQAQQEEQRERMQQQAVMHYMQQQQQQQQQQLGGPPTPAINTPVHFQSPPPVPGEVLKVQSYLENPTSYHLQQSQHQKVREYLSETYGNKFAAHISPAQGSPKPPPAASPGVRAGHVLSSSAGNSAPNSPMAMLHIGSNPERELDDVIDNIMRLDDVLGYINPEMQMPNTLPLSSSHLNVYSSDPQVTASLVGVTSSSCPADLTQKRELTDAESRALAKERQKKDNHNLIERRRRFNINDRIKELGMLIPKANDLDVRWNKGTILKASVDYIRRMQKDLQKSRELENHSRRLEMTNKQLWLRIQELEMQARVHGLPTTSPSGMNMAELAQQVVKQELPSEEGPGEALMLGAEVPDPEPLPALPPQAPLPLPTQPPSPFHHLDFSHSLSFGGREDEGPPGYPEPLAPGHGSPFPSLSKKDLDLMLLDDSLLPLASDPLLSTMSPEASKASSRRSSFSMEEGDVL

SEQ ID NO: 5 - DNA, 593 bp, Artificial Sequence
  Synthetic sequence
cgcgaatcaacctctggattacaaaatttctgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcaactcctttccgggacttacgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgactcggatctccctttgggccgcctccccgc

SEQ ID NO: 6 - DNA, 1800 bp, Artificial Sequence
  Synthetic sequence
ggcgcgccgacgcgcatgctcctctagactcgaggaattcggtaccccgggttcgaaatcgataagcttgatatcgaattcctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaactcctgacctcaaatgatccacccacctaggcctcccaaagtgcacagattacaggcgtgggccaccgcacctggccaaatttttaatttttttctagagatagggtcttactgtgttgcccaggctggtgtcaaactcctgggctcaagcagatcctcctgcctcagcttcccaaagtggtgggattataggtgtgagccactgcgcccagtcagtagccccctctttgcccctcactgagccctactggatgttcttggttgtgtgacagtttccccatctattaaacagaaacccctatagcagaggggaggatgaggttggaaaatcaggagcattgttattctattcttgtgggatcggggaagcagacatctgggtggatgtttggggaatgctgggctcagttgaggaagtaggggggcccctggggcttacagggactggaagctctgagctggccagagggatgttgcaatcctgccagggtcttgtctatgctgtccctttcacaaccatccccctaccgccaggctgacacgtggttgtgggggcacaaggccagccgaactagagtctgaggctgggctgaggacaccctccccatcagctgccagggtcactggcggtcaaaggcagctggtggggaaggaattggactccagccctgggggacggatgtggtgatggtgggaagcaggcttggtgccaggaggggcatcagagggtgaataagagcagatagagtgtttgggggaggtagccagccaaagggggtgaggcccggtggaagggaagaaggggcatacactcagagctttgcagctgaaggttttaattttttgagatggggtctcactctgtctcaccaggctggagtgcagtggcgcaatcacagctcactgcagcccgggggatccggagagctcgtcgacggcgcgcc

SEQ ID NO: 7 - DNA, 629 bp, Artificial Sequence
  Synthetic sequence
aattcctgcagcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcct

SEQ ID NO: 8 - DNA, 28285 bp, Artificial Sequence
  Synthetic sequence
aaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggtaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtatcacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaattttctggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcataggccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagatggcaccacttcactccagcctgggaaacagagcaagactctgtctctgagctgagatggcaccacttcactccagcctgggaaacagagcaagactctgtctcaaaaaaaacaaaacacacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactgggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactcccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagatttgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgtgtatatatacatgtatatatactgtgtatatatacatgtatatatactgtgtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccctttggggagtcctaagagggcagctggcaatggacacctagcagtccctttgagacttatttcagatggagctgtagaaagatgccatggctcacagtgcctccctgggaagggggcagagggctgcccagtgaggcctcttgcgagcaggaaatcaccagagacaaggaaagaccagaccccaggatgacctcagttaggccttgcccgactgtcctcagagtcccattctctgtgtcctggttcttttagaagatcatggacctccaggtcatttcgtaaccggaatctgcctgcggggggttttgacaagctatggtatagtgtatgtgggggtactgacgaattggaagatcatggagaccccttctcctcctccatcattggtctgccacatccctcccaggcgactcacagcagagagaccttggatgtatgtagggtgctttaaaactccagctgagttacagtctctcctttctgttttcaccttaaccttccagggatgcaaacccacgacaggtttagcagcagagtggaggctggccatgaatctcagagaaagtgctcactggaaaggctggtttagcccaggcctgatgtggaggcactgagctggacgttctagcggggttgacacccaacagtttacatagggggaggccacccctcctgagcagtctcggtgacttgaagaggaagccgcttcttctgtaccaacacagaagctccagcgaacccccagaatgctggcagtgtgggtgctatgtaaaagtatttacatagctttgtagagtgagccaagcccagtctgtttgggatgactcttcacagtgcctcgaatctgtcacacgtcttagtaagcagagtcacagagtttctgtcacatcatcctcctgcctacagggaagtaggccatgtccctgccccctactctgagcccagctgtgggagccagccctgcccaatgggctctctctgattggcttctcactcacttctaaactccagtgagcaacttctctcggctcgttcaattggcgtgaaggtctgtgtcttgcagagaaggttcttcacaactgggataaaggtctcgctgctcaagtgtagcccagtagaactgccaagccccttcccctcctctccctagactcttggatgcaagaagaatccaggcagctccaagggtgattgtgtccaacctagaatgtcttgaaaaagacattaaggggactagagaagacaggggatccaacggttctctgcagcccagcctgactgacatgtaactcttctggttctcaccagccagctggacctgcttagtattctttctgcctcagtttcccagcctgtacccagggctgtcatagttccatttcaggcagtagtaatgaatgagctgacataaaacatttagagcaggggtcagtatgtatatagagtgattattctatatcaggcattgcctcctcggaatgaagcttacaatcacccctccctctgcagttcatcttggggtggccagaggatccagcagacacctagtggggtaacacaccccagccaactcggctgttgcagactttgtctagaagtttcacgtctcagagctgaattcccttctcatgacctttggccgtgggagtgacacctcacagctgtggtgttttgacaaccagcagccactggcacacaaaatgtgcagccagcagcatatgaagtccaagaggcgtcccggccagccctgtccttgacccccacctgacaattaaggcaagagcctatagtttgcatcagcaacagtcacggtcaaagtttagtcaatcaaacgttgtgtaaggactcaactatggctgacacgggggcctgaggcctcccaacattcattaacaacagcaagttcaatcattatctccccaaagtttattgtgttaggtcagttccaaaccgtgctgaccatggctatgatccaaaggccggccccttacgtcagaggcgagcctccaggtccagctgaggggcagggctgtcctcccttctgtatactatttaaagcgaggagggctagctaccaagcacggttggccttccctctgggaacacacccttggccaacaggggaaatccggcgagacgctctgagatcctgcgagaaggaggtgcgtcctgctgcctgccccggcactctggctccccagctcaaggttcaggccttgccccaggccgggcctctgggtacctgaggtcttctcccgctctgtgcccttctcatggcgtcacgcatagggttgcgcatgcagctcatgcgggagcaggcgcagcaggaggagcagcgggagcgcatgcagcaacaggctgtcatgcattacatgcagcagcagcagcagcagcaacagcagcagctcggagggccgcccaccccggccatcaatacccccgtccacttccagtcgccaccacctgtgcctggggaggtgttgaaggtgcagtcctacctggagaatcccacatcctaccatctgcagcagtcgcagcatcagaaggtgcgggagtacctgtccgagacctatgggaacaagtttgctgcccacatcagcccagcccagggctctccgaaacccccaccagccgcctccccaggggtgcgagctggacacgtgctgtcctcctccgctggcaacagtgctcccaatagccccatggccatgctgcacattggctccaaccctgagagggagttggatgatgtcattgacaacattatgcgtctggacgatgtccttggctacatcaatcctgaaatgcagatgcccaacacgctacccctgtccagcagccacctgaatgtgtacagcagcgacccccaggtcacagcctccctggtgggcgtcaccagcagctcctgccctgcggacctgacccagaagcgagagctcacagatgctgagagcagggccctggccaaggagcggcagaagaaagacaatcacaacttaattgaaaggagacgaaggttcaacatcaatgaccgcatcaaggagttgggaatgctgatccccaaggccaatgacctggacgtgcgctggaacaagggcaccatcctcaaggcctctgtggattacatccggaggatgcagaaggacctgcaaaagtccagggagctggagaaccactctcgccgcctggagatgaccaacaagcagctctggctccgtatccaggagctggagatgcaggctcgagtgcacggcctccctaccacctccccgtccggcatgaacatggctgagctggcccagcaggtggtgaagcaggagctgcctagcgaagagggcccaggggaggccctgatgctgggggctgaggtccctgaccctgagccactgccagctctgcccccgcaagccccgctgcccctgcccacccagccaccatccccattccatcacctggacttcagccacagcctgagctttgggggcagggaggacgagggtcccccgggctaccccgaacccctggcgccggggcatggctccccattccccagcctgtccaagaaggatctggacctcatgctcctggacgactcactgctaccgctggcctctgatccacttctgtccaccatgtcccccgaggcctccaaggccagcagccgccggagcagcttcagcatggaggagggcgatgtgctgtgagaattccgcgaatcaacctctggattacaaaatttctgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcaactcctttccgggacttacgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgactcggatctccctttgggccgcctccccgcggcgcgccgacgcgcatgctcctctagactcgaggaattcggtaccccgggttcgaaatcgataagcttgatatcgaattcctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaactcctgacctcaaatgatccacccacctaggcctcccaaagtgcacagattacaggcgtgggccaccgcacctggccaaatttttaatttttttctagagatagggtcttactgtgttgcccaggctggtgtcaaactcctgggctcaagcagatcctcctgcctcagcttcccaaagtggtgggattataggtgtgagccactgcgcccagtcagtagccccctctttgcccctcactgagccctactggatgttcttggttgtgtgacagtttccccatctattaaacagaaacccctatagcagaggggaggatgaggttggaaaatcaggagcattgttattctattcttgtgggatcggggaagcagacatctgggtggatgtttggggaatgctgggctcagttgaggaagtaggggggcccctggggcttacagggactggaagctctgagctggccagagggatgttgcaatcctgccagggtcttgtctatgctgtccctttcacaaccatccccctaccgccaggctgacacgtggttgtgggggcacaaggccagccgaactagagtctgaggctgggctgaggacaccctccccatcagctgccagggtcactggcggtcaaaggcagctggtggggaaggaattggactccagccctgggggacggatgtggtgatggtgggaagcaggcttggtgccaggaggggcatcagagggtgaataagagcagatagagtgtttgggggaggtagccagccaaagggggtgaggcccggtggaagggaagaaggggcatacactcagagctttgcagctgaaggttttaattttttgagatggggtctcactctgtctcaccaggctggagtgcagtggcgcaatcacagctcactgcagcccgggggatccggagagctcgtcgacggcgcgccaattcctgcagcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctggcgcgcctaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttt

SEQ ID NO: 9 - protein, 394 aa, Homo sapiens
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

(19) United States
(12) Patent Application Publication
(10) Pub. No.: US 2015/0079051 A1
(43) Pub. Date: Mar. 19, 2015

(54) TFEB GENE THERAPY OF ALPHA-1-ANTITRYPSIN DEFICIENCY

(71) Applicant: FONDAZIONE TELETHON, Roma (RM) (IT)

(72) Inventors: Nicola Brunetti-Pierri, Napoli (NA) (IT); Andrea Ballabio, Napoli (NA) (IT); Nunzia Pastore, Napoli (NA) (IT)

(21) Appl. No.: 14/394,816

(22) PCT Filed: Apr. 19, 2013
(86) PCT No.: PCT/EP2013/058229

§ 371 Date: Oct. 16, 2014

(30) Foreign Application Priority Data
Apr. 19, 2012 (EP) 12164785

(51) Int. Cl. A61K 48/00; A61K 38/17

(52) U.S. Cl. CPC A61K 48/0058; A61K 38/1709
USPC 424/93.21; 514/44 R; 424/93.2; 514/20.4

(57) ABSTRACT

The present invention refers to a vector for gene therapy comprising a TFEB coding sequence under the control of a promoter able to efficiently express said TFEB coding sequence, to host cell comprising said vector and to their use in the gene therapy of a pathological condition characterized by a deficiency of alpha-1-antitrypsin (AAT). The present invention also refers to a pharmaceutical composition comprising the vector or the host cell of the invention for gene therapy and to a method for gene therapy of a pathological condition characterized by a deficiency of alpha-1-antitrypsin (AAT).

### TECHNICAL FIELD

The present invention relates to a vector comprising a TFEB coding sequence under the control of a promoter able to efficiently express said TFEB coding sequence for use in the gene therapy of a pathological condition characterized by a deficiency of alpha-1-antitrypsin (AAT), to a host cell comprising said vector, pharmaceutical compositions and uses thereof.

### BACKGROUND ART

Alpha-1-antitrypsin (AAT) is a member of the serine protease inhibitor (SERPIN) superfamily of structurally conserved proteins that inhibit serine proteases. AAT is synthesized in the liver and released into the plasma where it is the most abundant circulating protease inhibitor. AAT deficiency caused by missense mutation (lysine for glutamate at amino acid position 342) that alters protein folding is the most common genetic cause of liver disease in children′. It is also responsible for chronic liver disease and hepatocellular carcinoma in adults²⁻³. This mutated form of AAT is also known as ATZ. ATZ is prone to aggregate in the endoplasmic reticulum of hepatocytes causing liver injury. The only curative treatment available is liver transplantation. Therefore there is the need for alternative less invasive therapeutic strategies. Intracellular liver inclusions have been identified with other mutated form of AAT characterized by polymer formation, including S_{iiyama} (phenylalanine for serine at amino acid position 53) and Mₘₐₗₜₒₙ (deletion of phenylalanine at amino acid position 52)³⁴.

Recent findings have shown that stimulation of autophagy may reduce accumulation of hepatotoxic ATZ⁴⁻⁵. Transcription factor EB (TFEB, NCBI GeneID=7942; Accession no.: nt=NM_{—}007162.2, protein=NP_{—}009093.1 and variants thereof) is a master gene that regulates the number and function of lysosomes and autophagy by direct binding to a palindromic 10-base pair regulatory motif (CLEAR element) that is highly enriched in autophagy and lysosomal genes promoters⁶⁻⁸.

The document WO 2010/092112 relates to TFEB protein, synthetic or biotechnological functional derivative thereof, peptide fragments thereof, chimeric molecules comprising the TFEB protein, synthetic or biotechnological functional derivative thereof acting either direcly or indirectly on a CLEAR element. However the document is silent concerning pathological condition characterized by a deficiency of alpha-1-antitrypsin.

### SUMMARY OF THE INVENTION

In the present study, the authors investigated the therapeutic potential of liver-directed gene transfer of transcription factor EB (TFEB), a master gene that regulates lysosomal function and autophagy, in the PiZ transgenic mice, recapitulating the human hepatic disease. The authors have investigated efficiency of hepatic gene transfer of TFEB at increasing clearance of mutant hepatotoxic alpha-1-antitrypsin (ATZ). The authors injected a helper-dependent adenoviral (HDAd) vector expressing the TFEB under the control of a liver-specific promoter (HDAd-TFEB) in the PiZ mouse model, a transgenic mouse expressing the human ATZ gene and recapitulating the features of liver disease observed in humans. Three-month old PiZ mice were injected intravenously with HDAd-TFEB at the dose of 1×10¹³ vp/kg or, as controls, with the same dose of a HDAd vector expressing the unrelated alpha-fetoprotein (AFP) gene under the control of the same expression cassette (HDAd-AFP) or saline. Compared to saline or HDAd-AFP, mice injected with HDAd-TFEB showed a dramatic reduction in hepatic ATZ accumulation, as demonstrated by marked reduction of periodic acid-Schiff (PAS) staining and ATZ-containing globules. As expected, TFEB gene transfer resulted in an increase in hepatic LC3-II, a marker of autophagic activity. Taken together, these results demonstrate that hepatic gene transfer of TFEB reduces accumulation of ATZ by enhancement of autophagy in the liver. ATZ serum levels were reduced in mice injected with HDAd-TFEB vector as compared to baseline levels of the same mice. Moreover, a marked, statistically significant decrease in both ATZ monomer and polymer was observed in HDAd-TFEB injected mouse livers as compared to either saline or HDAd-AFP injected control mice, thus indicating that TFEB hepatic expression enhances disposal of both insoluble and soluble hepatic ATZ. In addition, the authors showed that HDAd-TFEB injected mice showed a reduction in hepatocyte apoptosis and hepatic fibrosis, which are key features of the hepatic disease of AAT deficiency. In summary, the authors showed that TFEB-mediated hepatocyte expression results in clearance of ATZ, improvement of the liver phenotype and therefore, is an attractive gene-based strategy for the treatment of alpha-1-antitrypsin deficiency hepatic disease.

Therefore, hepatocyte TFEB gene transfer resulted in a dramatic reduction of hepatic ATZ and reduced liver apoptosis and fibrosis, which are key features of AAT deficiency. Moreover, TFEB enhanced hepatic LC3, a marker of autophagy, and increased ATZ degradation by autophagolysosomes. For in vivo hepatocyte gene transfer, the authors used helper-dependent adenoviral (HDAd) vectors which are the most efficient vectors for liver-directed gene therapy⁹.

TFEB gene transfer is a novel strategy for treatment of liver disease of AAT deficiency. The present invention confirms the application of TFEB gene transfer for treatment of a wide spectrum of human disorders due to accumulation of toxic proteins.

In the present invention TFEB coding sequence means a sequence coding for the entire TFEB protein or for TFEB functional fragment(s), said fragment(s) preferably being able to act on a CLEAR element and having a transcription activation domain, and/or maintaining the clearance activity.

Object of the present invention is a vector comprising a TFEB coding sequence under the control of a promoter able to efficiently express said TFEB coding sequence for use in the gene therapy of a pathological condition characterized by a deficiency of alpha-1-antitrypsin (AAT).

Preferably the TFEB coding sequence is hTFEB consisting essentially of the sequence of SEQ ID No. 3.

In a preferred embodiment the vector is a viral vector. Preferably the viral vector belongs to the group of: adenoviral vectors, lentiviral vectors, retroviral vectors, Adeno associated vectors (AAV) or naked plasmid DNA vectors.

Still preferably the vector belongs to the group of helper-dependent adenoviral vectors.

In a preferred embodiment the deficiency of AAT is due to a mutation of the AAT gene.

Preferably the mutation of the AAT gene causes a substitution of glutamate into lysine at amino acid position 342 and/or a substitution of serine into phenylalanine at amino acid position 53 (S_{iiyama}) and/or a deletion of phenylalanine at amino acid position 52 (Mₘₐₗₜₒₙ) of the AAT protein (SEQ ID No. 9).

Still preferably the AAT deficiency is characterized by an accumulation of a wild type and/or mutated AAT protein in a tissue.

Yet preferably the accumulation of the wild type and/or mutated AAT protein further comprises the formation of wild type and/or mutated AAT aggregates in the tissue.

Preferably the tissue is liver.

Still preferably, the vector as described above comprises a liver specific promoter and, optionally, regulatory sequences.

Preferably the liver specific promoter is phosphoenolpyruvate carboxykinase (PEPCK) promoter consisting essentially of the sequence of SEQ ID No. 1.

Still preferably the liver regulatory sequence is the liver specific enhancer Locus Control Region (LCR) from the apoE locus consisting essentially of the sequence of SEQ ID No. 6.

In a preferred embodiment the vector of the invention comprises essentially the nucleotide sequence of SEQ ID No. 8.

It is a further object of the invention a host cell transformed by the vector as defined above.

It is a further object of the invention a viral particle containing the vector as defined above.

It is a further object of the invention a pharmaceutical composition comprising the vector of the invention or the host cell of the invention or the viral particle of the invention for use in the gene therapy of a pathological condition characterized by a deficiency of alpha-1-antitrypsin (AAT).

It is a further object of the invention a method for gene therapy of a pathological condition characterized by a deficiency of alpha-1-antitrypsin (AAT) of a subject in need thereof, said method comprising administering a suitable amount of the pharmaceutical composition as defined above.

It is a further object of the invention the TFEB protein, synthetic or biotechnological functional derivative thereof, peptide fragments thereof, chimeric molecules comprising the TFEB protein, synthetic or biotechnological functional derivative thereof for use in the treatment of a pathological condition characterized by a deficiency of alpha-1-antitrypsin (AAT).

Preferably, the TFEB protein consists essentially of the amino acid sequence of SEQ ID No. 4.

In the present invention a synthetic or biotechnological functional derivative of a protein, peptide fragments of a protein, chimeric molecules comprising the TFEB protein, synthetic or biotechnological functional derivative thereof are defined as molecules able to maintain the therapeutic effect of TFEB, i.e. the treatment of a pathological condition characterized by a deficiency of alpha-1-antitrypsin (AAT).

In the present invention, the viral vector may be selected from the group of: adenoviral vectors, adeno-associated viral (AAV) vectors, pseudotyped AAV vectors, herpes viral vectors, retroviral vectors, lentiviral vectors, baculoviral vectors. Pseudotyped AAV vectors are those which contain the genome of one AAV serotype in the capsid of a second AAV serotype; for example an AAV2/8 vector contains the AAV8 capsid and the AAV 2 genome³⁵. Such vectors are also known as chimeric vectors. Naked plasmid DNA vectors and other vectors known in the art may be used to deliver a TFEB gene according to the present invention³⁶. Other examples of delivery systems include ex vivo delivery systems, which include but are not limited to DNA transfection methods such as electroporation, DNA biolistics, lipid-mediated transfection, compacted DNA-mediated transfection. Typically, a viral vector can accommodate a transgene (i.e., a TFEB gene described herein) and regulatory elements.

Various methods may be used to deliver viral vectors encoding a TFEB gene described herein into a subject in need of treatment. For example, a viral vector may be delivered through intravenous or intravascular injection. Other routes of systemic administration include, but are not limited to, intra-arterial, intra-cardiac, intraperitoneal and subcutaneous or via local administration such as muscle injection or intramuscular administration.

The vector of the invention, in particular the HDAd vector may be injected at a dose range between 1×10e10 viral particles (vp)/kg and 1×10e13 vp/kg.

A dose range between 1×10e11 and 1×10e12 vp/kg is more likely to be effective in humans because these doses are expected to result in large transduction efficiency of the liver.

The vector of the invention, in particular the AAV vector may be injected at doses between 1×10e11 vector genomes (vg)/kg and 1×10e13 vg/kg are expected to provide high liver transduction²⁶.

Adenoviral vector genomes do not integrate into the genome of the transduced cells and therefore vector genomes are lost in actively dividing cells³⁷. Should TFEB expression fade over time, to maintain phenotypic correction it would be possible to re-administer a vector with a different serotype to overcome the neutralizing anti-Ad antibody elicited with the first administration^{38,39}.

The present invention provides pharmaceutical compositions comprising: a) an effective amount of a vector as described herein or an effective amount of a transformed host cell as described herein, and b) a pharmaceutically acceptable carrier, which may be inert or physiologically active.

As used herein, “pharmaceutically-acceptable carriers” includes any and all solvents, dispersion media, coatings, antibacterial and antifungal agents, and the like that are physiologically compatible. Examples of suitable carriers, diluents and/or excipients include one or more of water, saline, phosphate buffered saline, dextrose, glycerol, ethanol, and the like, as well as combination thereof. In many cases, it will be preferable to include isotonic agents, such as sugars, polyalcohols, or sodium chloride in the composition. In particular, relevant examples of suitable carrier include: (1) Dulbecco's phosphate buffered saline, pH˜7.4, containing or not containing about 1 mg/ml to 25 mg/ml human serum albumin, (2) 0.9% saline (0.9% w/v sodium chloride (NaCl)), and (3) 5% (w/v) dextrose; and may also contain an antioxidant such as tryptamine and a stabilizing agent such as Tween 20.

The pharmaceutical compositions encompassed by the present invention may also contain a further therapeutic agent for the treatment of a pathological condition characterized by a deficiency of alpha-1-antitrypsin (AAT).

The compositions of the invention may be in a variety of forms. These include for example liquid, semi-solid, but the preferred form depends on the intended mode of administration and therapeutic application. Typical preferred compositions are in the form of injectable or infusible solutions. The preferred mode of administration is parenteral (e.g. intravenous, intramuscular, intraperinoneal, subcutaneous). In a preferred embodiment, the compositions of the invention are administered intravenously as a bolus or by continuous infusion over a period of time. In another preferred embodiment, they are injected by intramuscular, subcutaneous, intraarticular, intrasynovial, intratumoral, peritumoral, intralesional, or perilesional routes, to exert local as well as systemic therapeutic effects.

Sterile compositions for parenteral administration can be prepared by incorporating the vector or host cell as described in the present invention in the required amount in the appropriate solvent, followed by sterilization by micro filtration. As solvent or vehicle, there may be used water, saline, phosphate buffered saline, dextrose, glycerol, ethanol, and the like, as well as combination thereof. In many cases, it will be preferable to include isotonic agents, such as sugars, polyalcohols, or sodium chloride in the composition. These compositions may also contain adjuvants, in particular wetting, isotonizing, emulsifying, dispersing and stabilizing agents. Sterile compositions for parenteral administration may also be prepared in the form of sterile solid compositions which may be dissolved at the time of use in sterile water or any other injectable sterile medium.

There may be used pharmaceutically acceptable solutions, suspensions, emulsions, syrups and elixirs containing inert diluents such as water, ethanol, glycerol, vegetable oils or paraffin oil. These compositions may comprise substances other than diluents, for example wetting, sweetening, thickening, flavoring or stabilizing products.

The doses depend on the desired effect, the duration of the treatment and the route of administration used and may be determined easiy by the skilled person in the art using known methods.

As well known in the medical arts, dosages for any one patient depends upon many factors, including the patient's size, body surface area, age, the particular compound to be administered, sex, time and route of administration, general health, and other drugs being administered concurrently.

The present invention will be illustrated by means of non-limiting examples referring to the following figures.

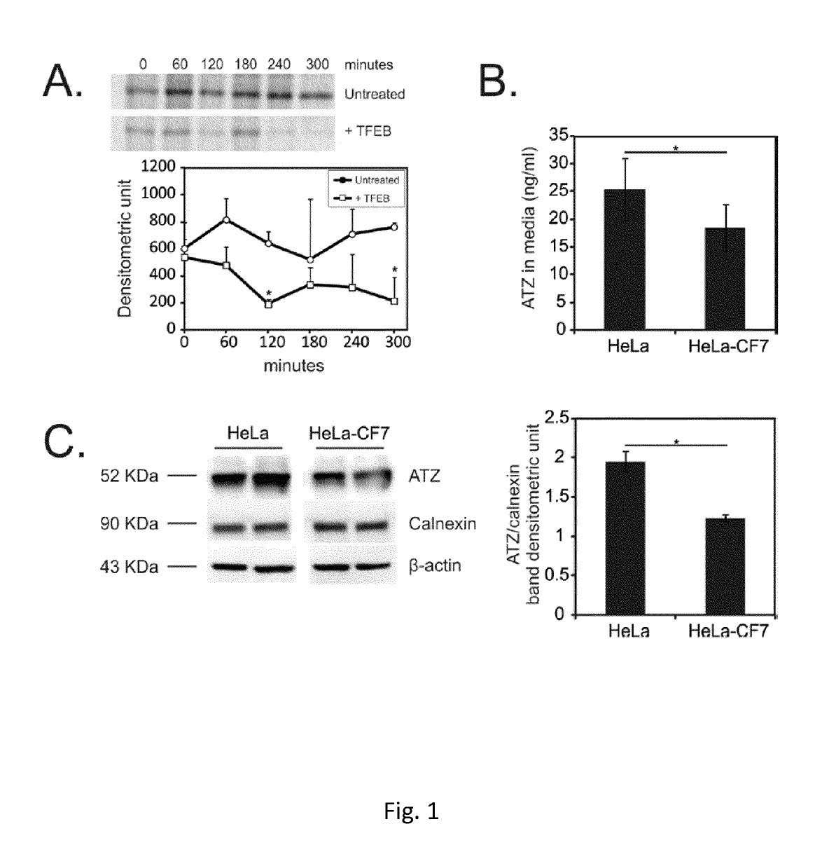

FIG. 1. (A) ATZ stably transfected mouse Hepa1,6 cell lysates were immunoprecipitated with anti-AAT antibody (which recognize also ATZ) following pulse labeling. The intracellular, newly synthesized ATZ decreased more rapidly in TFEB transfected cells as compared to control untreated cells. Kinetics of disappearance of intracellular ATZ determined by densitometric analysis from 2 independent experiments showed statistical significant reduction at 120 and 300 minutes of chase (*p<0.05). HeLa cells stably over-expressing TFEB (HeLa-CF7) secrete reduced amount of ATZ in the media (B) and accumulate less ATZ intracellularly (C) 24 hours after transfection with a plasmid expressing ATZ. Two independent samples from HeLa and HeLa-CF7 cells are shown. Media ATZ and ELISA and band quantifications were performed on n=3 per condition (*p<0.05).

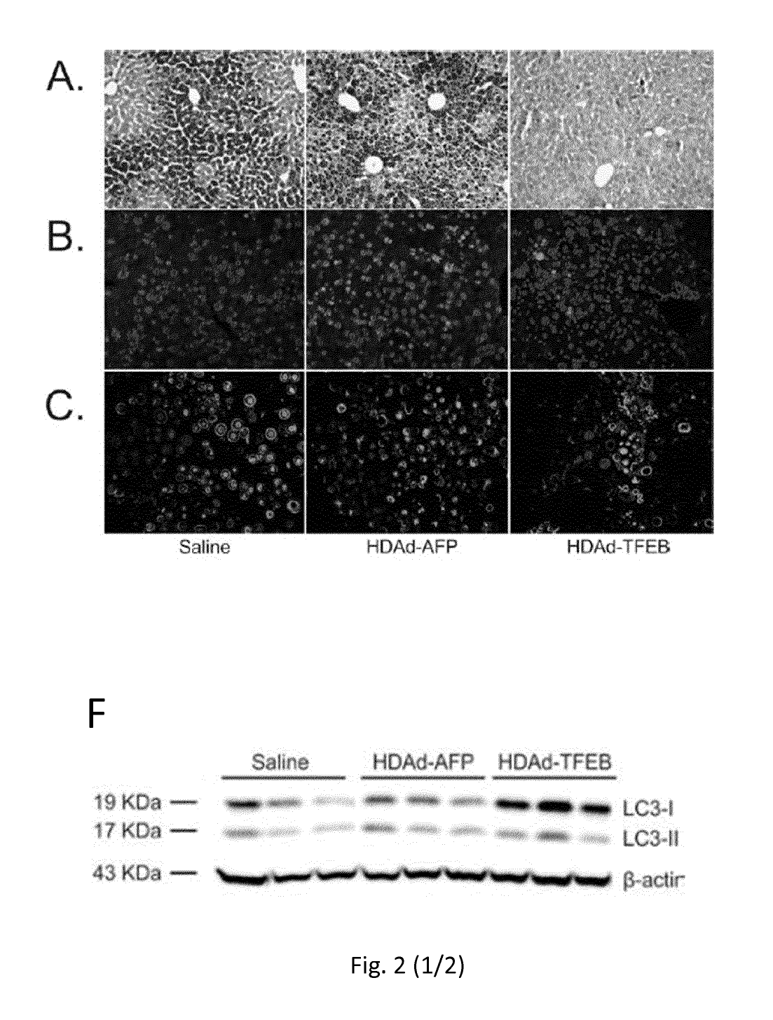

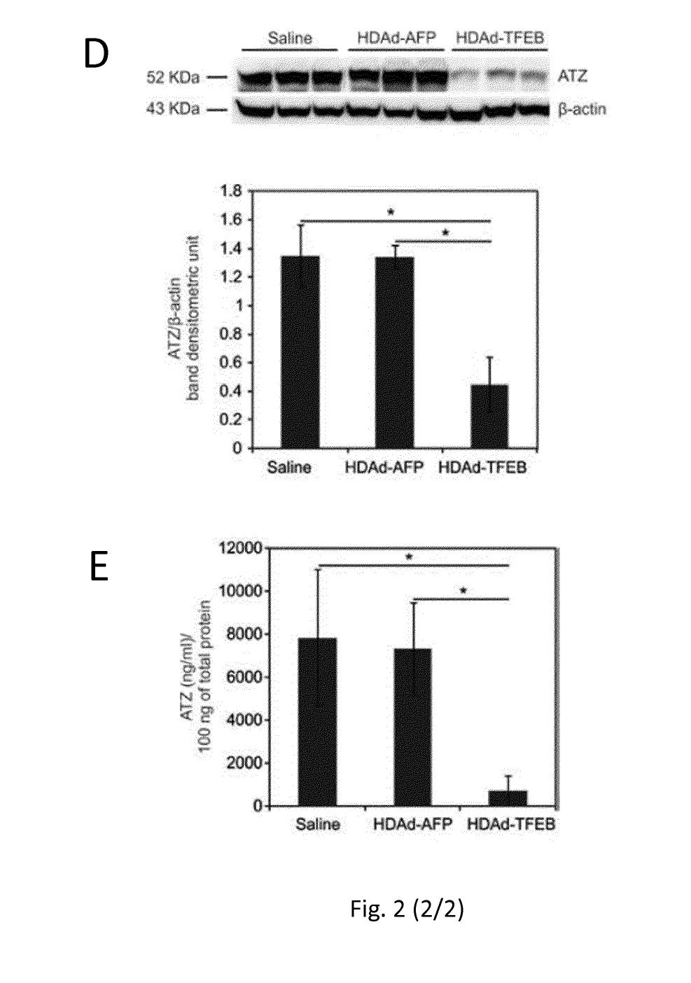

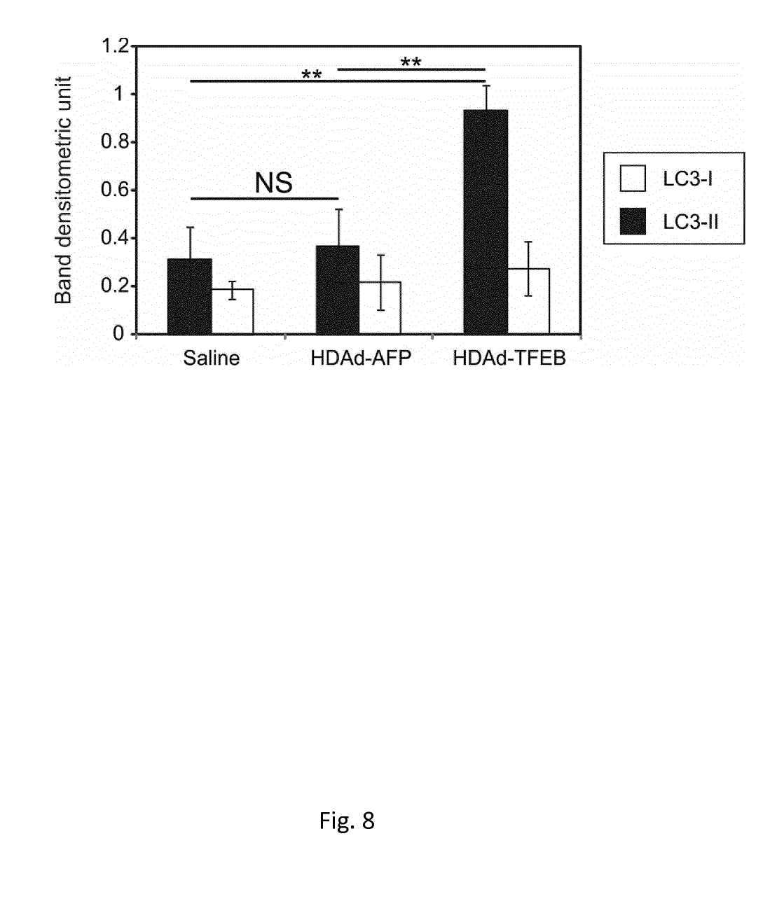

FIG. 2. (A) PAS staining was markedly reduced in livers from mice injected with HDAd-TFEB as compared to saline or HDAd-AFP injected mice (magnification 20×). (B) Immunofluorescence of livers from HDAd-TFEB injected mice showed a marked reduction of ATZ globules as compared to controls (magnification 20×). (C) Co-staining of liver specimens for LAMP-1 (red) and ATZ (green) shows an increase in LAMP-1 staining in HDAd-TFEB injected mice. For HDAd-TFEB injected livers the selected field presented shows a region of increased LAMP-1 signal and absent ATZ surrounding an area of increased ATZ staining (magnification 63×). (D) Western blot analysis show a significant reduction in ATZ band intensities in livers of HDAd-TFEB injected PiZ mice as compared to HDAd-AFP and saline injected controls. Three of five representative mice are shown in the western blot analysis. Quantification of band intensities was performed on n=5 mice per group (*p<0.05). (E) ELISA for ATZ on hepatic extracts show a statistically significant (*p<0.05) reduction in the amount of ATZ in HDAd-TFEB injected mice (n=5 per group). (F) Western blot for LC-3 showed an increase in LC3-I in mice injected with HDAd-TFEB as compared to controls. Quantification of band intensities is shown in FIG. 8.

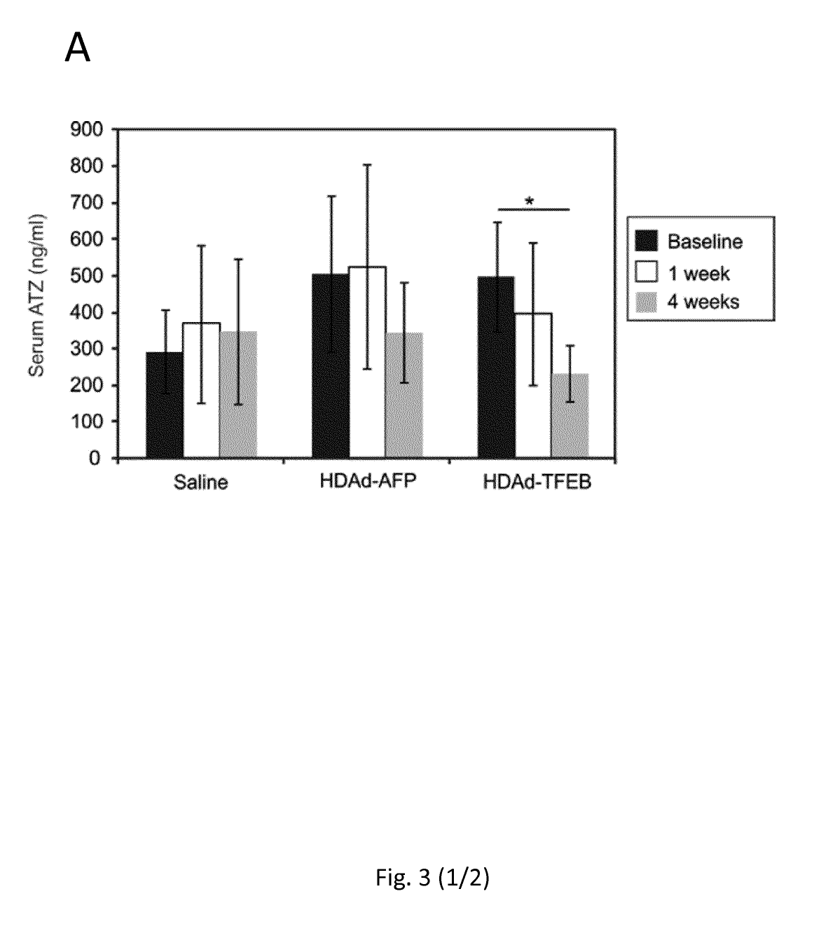

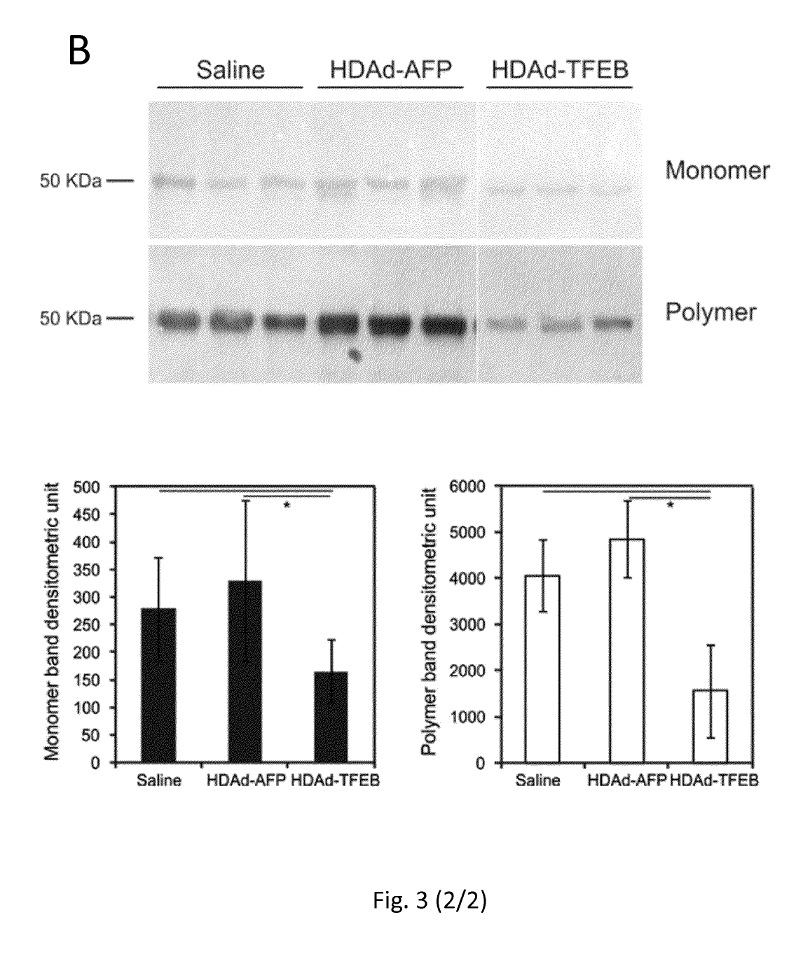

FIG. 3. (A) Effect of hepatocyte TFEB expression on serum levels of human ATZ in PiZ mice. Serum levels were determined by ELISA specific for human AAT (n=5 per group; *p<0.05). (B) Monomer-polymer analysis of mouse livers. Representative bands from 3 mice for each treatment group are shown. The graph shows densitometric quantification of n=5 mice from each treatment group. A marked, statistically significant decrease in both ATZ monomer and polymer was observed in HDAd-TFEB injected mouse livers as compared to either saline or HDAd-AFP injected control mice (*p<0.05).

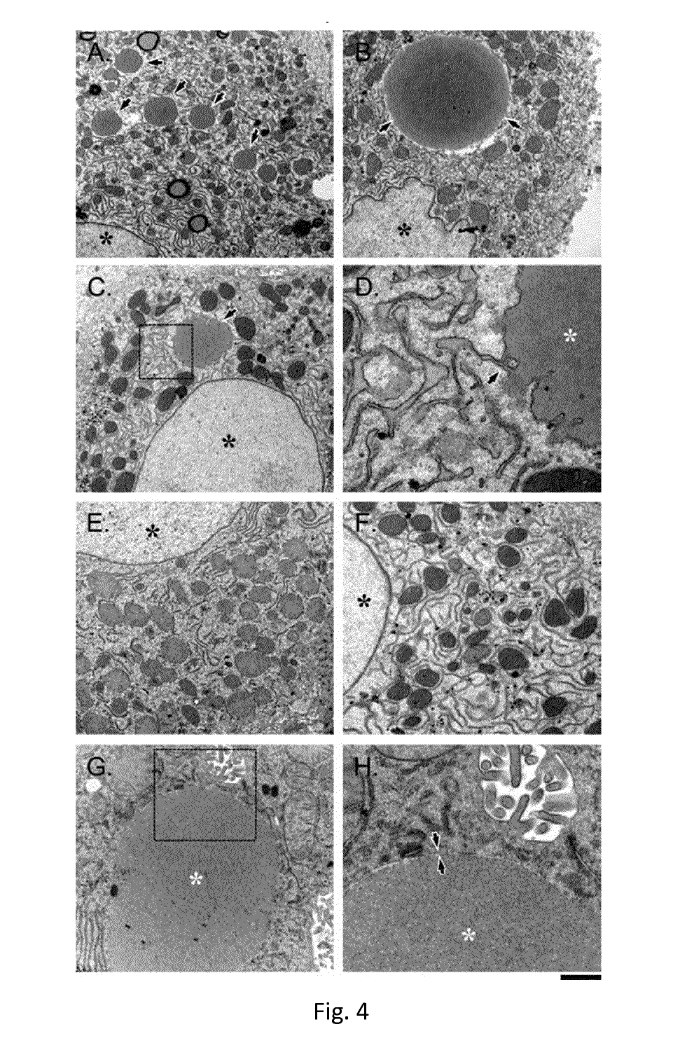

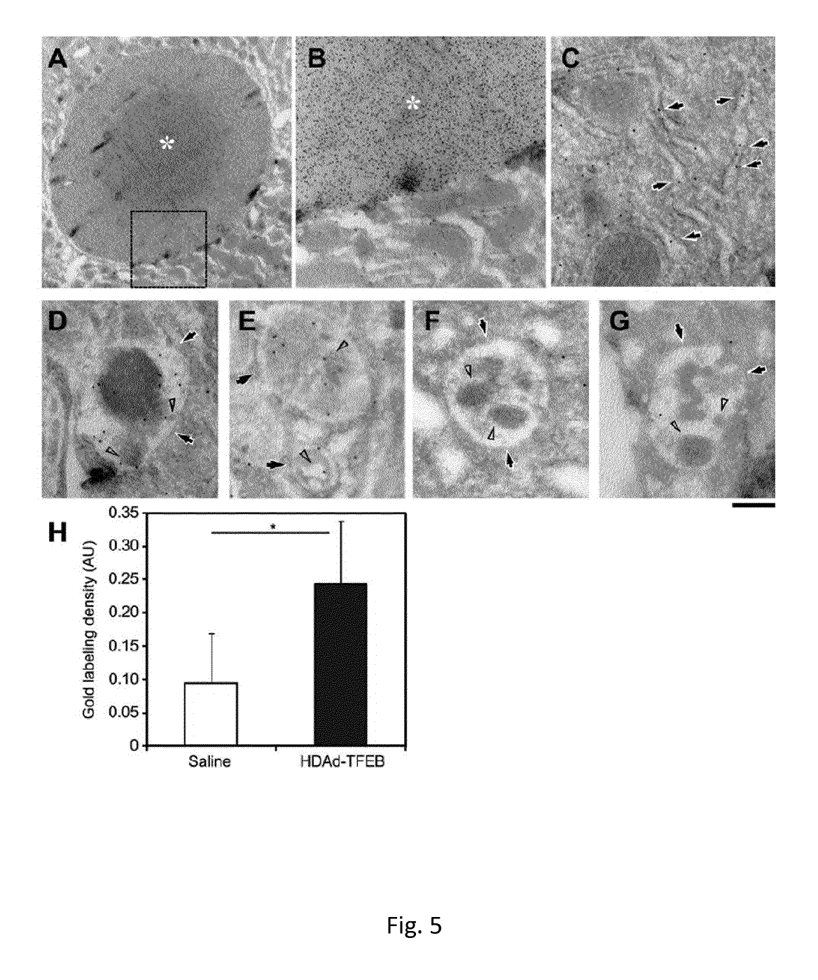

FIG. 4. Livers from either saline injected (A-D) or HDAd-TFEB injected (E-H) mice were prepared for EM as described in online Methods. (A-C) Arrows show membrane bound inclusions in the hepatocyte cytoplasm, while asterisks indicate nuclei. (D) This panel shows the area corresponding to the region outlined by dash box in C. Arrow indicates the place where inclusion (asterisk) is connected with RER membranes. (E, F) Hepatocytes in HDAd-TFEB injected animals do not exhibit inclusions in cytoplasm. Asterisks indicate nuclei. (G) Example of hepatocyte that still contains inclusion (asterisk). (H) The image shows the area corresponding to the region outlined by dash box in G. Arrows indicate double membrane around inclusion (asterisk). Scale bar: 1.5 μm (A-C), 340 nm (D), 1.3 μm (E, F), 950 nm (G), 380 nm (H) FIG. 5. Livers from either saline-injected (A, B, F, G) or HDAd-TFEB injected (C-E) mice were prepared for immuno-EM of ATZ as described in online Methods. (A) Region of the hepatocyte cytoplasm exhibits large membrane bound inclusion (asterisk) similar to that shown in FIG. 4B. (B) Higher magnification from the region outlined by dash box in A reveals dense gold ATZ labeling over inclusion body (asterisk). (C) Arrows indicate diffuse ATZ signal along the RER membranes of hepatocyte in HDAd-TFEB-treated mice. (D, G) Liver cells from HDAd-TFEB-injected mice exhibit ATZ-corresponding gold particles within the lysosome-like structures (arrows) where ATZ signal is frequently associated with intraluminal vesicles (arrowheads). (E, F) Lysosomes (asterisks) and their intraluminal vesicles (arrowheads) show little or no ATZ in control animals. (H) Immuno-gold ATZ labeling densities (n=20 cells) in lysosome-like structures were quantified as described in online Methods and expressed in arbitrary units (AU). Scale bar: 1.8 μm (A), 520 nm (B), 320 nm (C), 250 nm (D), 220 nm (E-G).

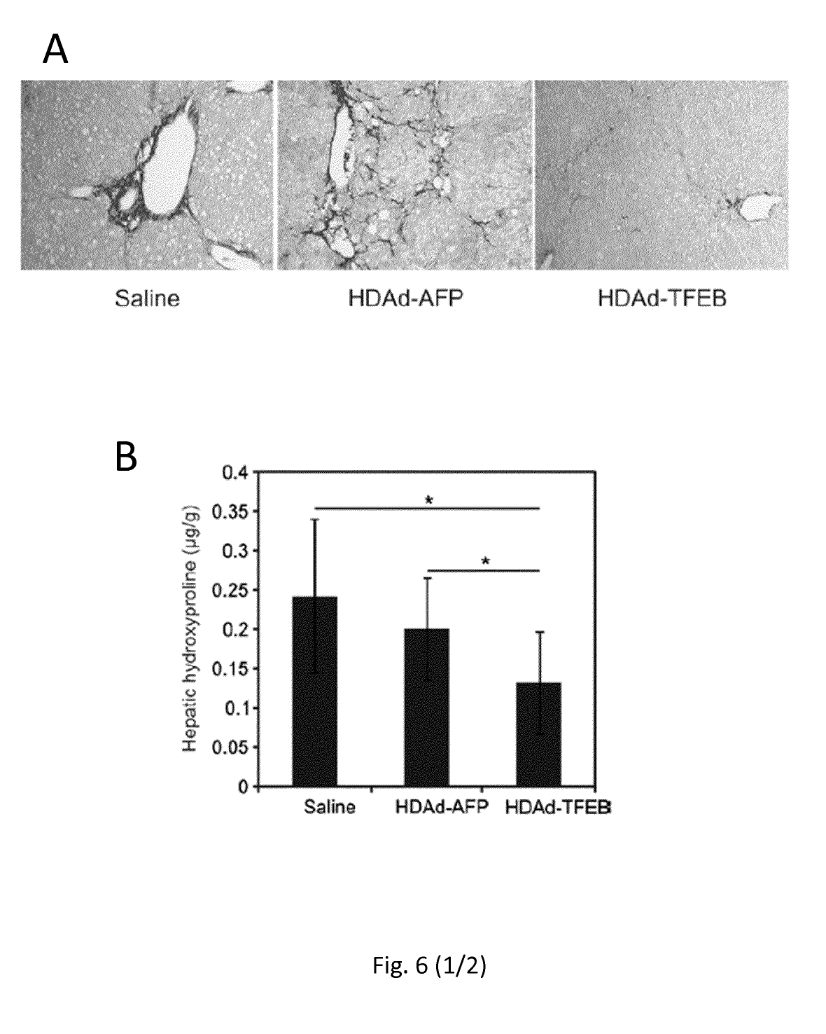

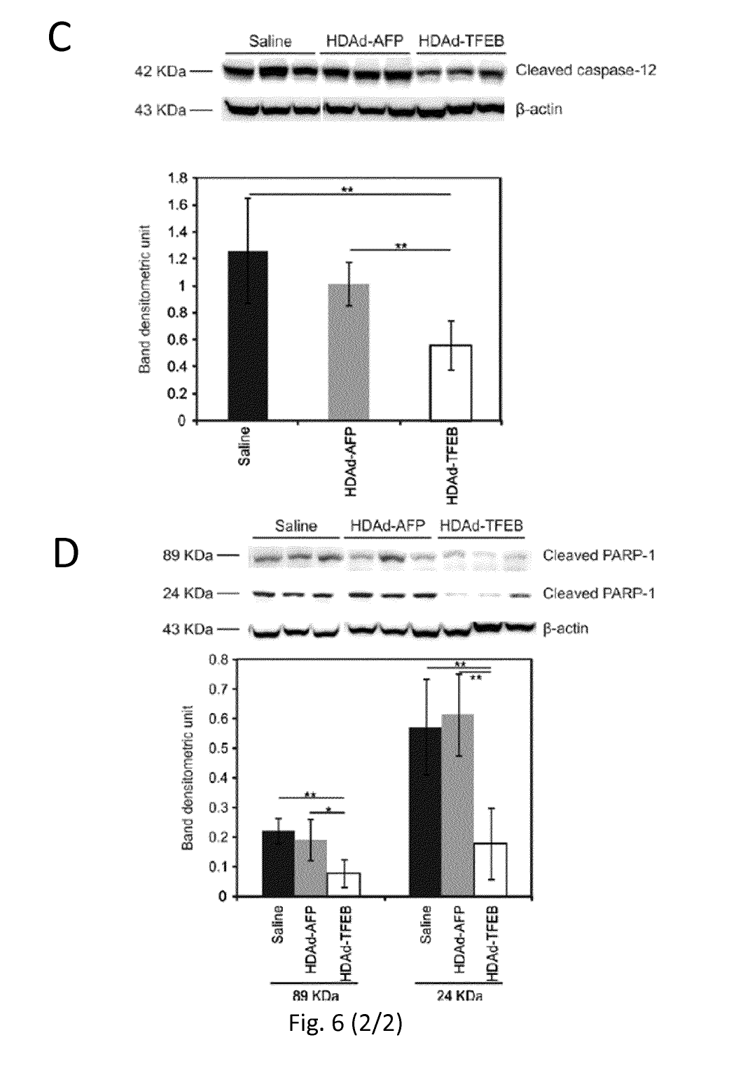

FIG. 6. Sirius red staining (A) (magnification 20×) and hepatic hydroxyproline content (B) showed a reduction of fibrosis in PiZ mice injected with HDAd-TFEB compared to saline ore HDAd-AFP injected mice (n=5 per group; *p<0.05). (C) Western blot analysis showed that PiZ mice injected with HDAd-TFEB have a significant reduction of cleaved caspase-12 compared to saline or HDAd-AFP injected mice (representative bands from 3 independent animals are shown). Band densities of cleaved 42 KDa caspase-12 band normalized for β-actin levels showed a statistical significant (*p<0.05) reduction in HDAd-TFEB injected mice (n=5 per group). (D) Western blot analysis showed that PiZ mice injected with HDAd-TFEB have a significant reduction of cleaved PARP-1 compared to saline or HDAd-AFP injected mice (representative bands from 3 independent animals are shown). Densities of cleaved 89 and 24 KDa PARP-1 bands normalized for β-actin levels showed a statistical significant reduction in HDAd-TFEB injected mice (n=5 per group; *p<0.05, **p<0.01).

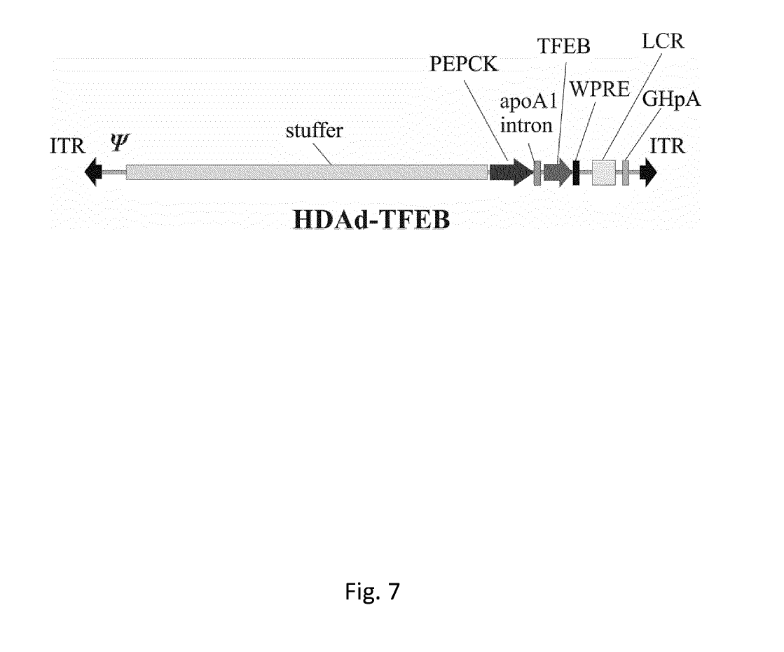

FIG. 7. HDAd-TFEB vector. ITR means sequences necessary for virus replication. ψ means the packaging sequence that ensures packaging of the vector DNA into virions. “stuffer” means human genomic DNA sequences or other non-transcribed DNA sequences used to increase the vector insert size. “WPRE” means the mRNA-stabilizing post-transcriptional regulatory element from the woodchuck hepatitis virus. “BGHpA” means the bovine growth hormone polyadenylation signal. This sequence represents a specialized termination sequence for protein expression in eukaryotic cells. HDAd-TFEB contains the human TFEB transgene under the control of a liver-specific PEPCK promoter. The expression cassette contains the ApoAI intron, the woodchuck hepatitis post-transcriptional regulatory element (WPRE), the Locus Control Region (LCR) from the apoE locus and the human growth hormone poly A (GHpA). Adenoviral inverted terminal repeats (ITR) and packaging signal (Ψ) are shown. Not drawn to scale.

FIG. 8. Quantification of LC3 in HDAd-TFEB injected mice. Densitometric analysis of LC3 bands on Western blot performed on n=5 mice per group. No change in LC3-II was detected while LC3-I was increased in HDAd-TFEB injected mouse livers. **p<0.01; NS=not statistically significant.

SEQUENCES:
PEPCK PROMOTER
(SEQ ID No. 1)
ctttggggagtcctaagagggcagctggcaatggacacctagcagtccctttgagacttatttcagatggagctgtagaaagatgccatggc
tcacagtgcctccctgggaagggggcagagggctgcccagtgaggcctcttgcgagcaggaaatcaccagagacaaggaaagaccag
accccaggatgacctcagttaggccttgcccgactgtcctcagagtcccattctctgtgtcctggttcttttagaagatcatggacctccaggtc
atttcgtaaccggaatctgcctgcggggggttttgacaagctatggtatagtgtatgtgggggtactgacgaattggaagatcatggagaccc
cttctcctcctccatcattggtctgccacatccctcccaggcgactcacagcagagagaccttggatgtatgtagggtgctttaaaactccagc
tgagttacagtctctcctttctgttttcaccttaaccttccagggatgcaaacccacgacaggtttagcagcagagtggaggctggccatgaat
ctcagagaaagtgctcactggaaaggctggtttagcccaggcctgatgtggaggcactgagctggacgttctageggggttgacacccaa
cagtttacatagggggaggccacccctcctgagcagtctcggtgacttgaagaggaagccgcttcttctgtaccaacacagaagctccagc
gaacccccagaatgctggcagtgtgggtgctatgtaaaagtatttacatagctttgtagagtgagccaagcccagtctgtttgggatgactctt
cacagtgcctcgaatctgtcacacgtcttagtaagcagagtcacagagtttctgtcacatcatcctcctgcctacagggaagtaggccatgtc
cctgccccctactctgagcccagctgtgggagccagccctgcccaatgggctctctctgattggcttctcactcacttctaaactccagtgag
caacttctctcggctcgttcaattggcgtgaaggtctgtgtcttgcagagaaggttcttcacaactgggataaaggtctcgctgctcaagtgta
gcccagtagaactgccaagccccttcccctcctctccctagactcttggatgcaagaagaatccaggcagctccaagggtgattgtgtccaa
cctagaatgtcttgaaaaagacattaaggggactagagaagacaggggatccaacggttctctgcagcccagcctgactgacatgtaactct
tctggttctcaccagccagctggacctgettagtattctttctgcctcagtttcccagcctgtacccagggctgtcatagttccatttcaggcagt
agtaatgaatgagctgacataaaacatttagagcaggggtcagtatgtatatagagtgattattctatatcaggcattgcctcctcggaatgaag
cttacaatcacccctccctctgcagttcatcttggggtggccagaggatccagcagacacctagtggggtaacacaccccagccaactcgg
ctgttgcagactttgtctagaagtttcacgtctcagagctgaattcccttctcatgacctttggccgtgggagtgacacctcacagctgtggtgtt
ttgacaaccagcagccactggcacacaaaatgtgcagccagcagcatatgaagtccaagaggcgtcccggccagccctgtccttgacccc
cacctgacaattaaggcaagagcctatagtttgcatcagcaacagtcacggtcaaagtttagtcaatcaaacgttgtgtaaggactcaactatg
gctgacacgggggcctgaggcctcccaacattcattaacaacagcaagttcaatcattatctccccaaagtttattgtgttaggtcagttccaa
accgtgctgaccatggctatgatccaaaggccggccccttacgtcagaggcgagcctccaggtccagctgaggggcagggctgtcctcc
cttctgtatactatttaaagcgaggagggctagctaccaagcacggttggccttccctctgggaacacacccttggccaacaggggaaatcc
ggcgagacgctctgag
APOA1 INTRON
(SEQ ID No. 2)
atcctgcgagaaggaggtgcgtcctgctgcctgccccggcactctggctccccagctcaaggttcaggccttgccccaggccgggcctct
gggtacctgaggtcttctcccgctctgtgcccttctc
HUMAN TFEB (NCBI GeneID = 7942; Accession no.: nt = NM_007162.2, protein = NP_009093.1)
(SEQ ID No. 3)
atggcgtcacgcatagggttgcgcatgcagctcatgcgggagcaggcgcagcaggaggagcagcgggagcgcatgcagcaacaggct
gtcatgcattacatgcagcagcagcagcagcagcaacagcagcagctcggagggccgcccaccccggccatcaatacccccgtccactt
ccagtcgccaccacctgtgcctggggaggtgttgaaggtgcagtcctacctggagaatcccacatcctaccatctgcagcagtcgcagcat
cagaaggtgcgggagtacctgtccgagacctatgggaacaagtttgctgcccacatcagcccagcccagggctctccgaaacccccacc
agccgcctccccaggggtgcgagctggacacgtgctgtcctcctccgctggcaacagtgctcccaatagccccatggccatgctgcacatt
ggctccaaccctgagagggagttggatgatgtcattgacaacattatgcgtctggacgatgtccttggctacatcaatcctgaaatgcagatg
cccaacacgctacccctgtccagcagccacctgaatgtgtacagcagcgacccccaggtcacagcctccctggtgggcgtcaccagcag
ctcctgccctgcggacctgacccagaagcgagagctcacagatgctgagagcagggccctggccaaggagcggcagaagaaagacaa
tcacaacttaattgaaaggagacgaaggttcaacatcaatgaccgcatcaaggagttgggaatgctgatccccaaggccaatgacctggac
gtgcgctggaacaagggcaccatcctcaaggcctctgtggattacatccggaggatgcagaaggacctgcaaaagtccagggagctgga
gaaccactctcgccgcctggagatgaccaacaagcagctctggctccgtatccaggagctggagatgcaggctcgagtgcacggcctcc
ctaccacctccccgtccggcatgaacatggctgagctggcccagcaggtggtgaagcaggagctgcctagcgaagagggcccagggga
ggccctgatgctgggggctgaggtccctgaccctgagccactgccagctctgcccccgcaagccccgctgcccctgcccacccagccac
catccccattccatcacctggacttcagccacagcctgagctttgggggcagggaggacgagggtcccccgggctaccccgaacccctg
gcgccggggcatggctccccattccccagcctgtccaagaaggatctggacctcatgctcctggacgactcactgctaccgctggcctctg
atccacttctgtccaccatgtcccccgaggcctccaaggccagcagccgccggagcagcttcagcatggaggagggcgatgtgctgtga
HUMAN TFEB protein
(SEQ ID No. 4)
MASRIGLRMQLMREQAQQEEQRERMQQQAVMHYMQQQQQQQQQQLGGPPTPAINTP
VHFQSPPPVPGEVLKVQSYLENPTSYHLQQSQHQKVREYLSETYGNKFAAHISPAQGSP
KPPPAASPGVRAGHVLSSSAGNSAPNSPMAMLHIGSNPERELDDVIDNIMRLDDVLGYI
NPEMQMPNTLPLSSSHLNVYSSDPQVTASLVGVTSSSCPADLTQKRELTDAESRALAKE
RQKKDNHNLIERRRRFNINDRIKELGMLIPKANDLDVRWNKGTILKASVDYIRRMQKDL
QKSRELENHSRRLEMTNKQLWLRIQELEMQARVHGLPTTSPSGMNMAELAQQVVKQE
LPSEEGPGEALMLGAEVPDPEPLPALPPQAPLPLPTQPPSPFHHLDFSHSLSFGGREDEGP
PGYPEPLAPGHGSPFPSLSKKDLDLMLLDDSLLPLASDPLLSTMSPEASKASSRRSSFSME
EGDVL
WPRE
(SEQ ID No. 5)
cgcgaatcaacctctggattacaaaatttctgaaagattgactggtattcttaactatgttgctccttttacgctatgtggatacgctgctttaat
gcctttgtatcatgctattgcttcccgtatggctttcattttctcctccttgtataaatcctggttgctgtctatttatgaggagttgtggcccgt
tgtcaggcaacgtggcgtggtgtgcactgtgtttgctgacgcaacccccactggttggggcattgccaccacctgtcaactcctttccgggactta
cgctttccccctccctattgccacggcggaactcatcgccgcctgccttgcccgctgctggacaggggctcggctgttgggcactgacaattccgt
ggtgttgtcggggaagctgacgtcctttccatggctgctcgcctgtgttgccacctggattctgcgcgggacgtccttctgctacgtcccttcg
gccctcaatccagcggaccttccttcccgcggcctgctgccggctctgcggcctcttccgcgtcttcgccttcgccctcagacgactcggat
ctccctttgggccgcctccccgc
LCR
(SEQ ID No. 6)
ggcgcgccgacgcgcatgctcctctagactcgaggaattcggtaccccgggttcgaaatcgataagcttgatatcgaattcctgcaggctca
gaggcacacaggagtttctgggctcaccctgcccccttccaacccctcagttcccatcctccagcagctgtttgtgtgctgcctctgaagtcc
acactgaacaaacttcagcctactcatgtccctaaaatgggcaaacattgcaagcagcaaacagcaaacacacagccctccctgcctgctg
accttggagctggggcagaggtcagagacctctctgggcccatgccacctccaacatccactcgaccccttggaatttcggtggagagga
gcagaggttgtcctggcgtggtttaggtagtgtgagagggtccgggttcaaaaccacttgctgggtggggagtcgtcagtaagtggctatgc
cccgaccccgaagcctgtttccccatctgtacaatggaaatgataaagacgcccatctgatagggtttttgtggcaaataaacatttggttttttt
gttttgttttgttttgttttttgagatggaggtttgctctgtcgcccaggctggagtgcagtgacacaatctcatctcaccacaaccttcccctgc
ctcagcctcccaagtagctgggattacaagcatgtgccaccacacctggctaattttctatttttagtagagacgggtttctccatgttggtcagc
ctcagcctcccaagtaactgggattacaggcctgtgccaccacacccggctaattttttctatttttgacagggacggggtttcaccatgttggtc
aggctggtctagaactcctgacctcaaatgatccacccacctaggcctcccaaagtgcacagattacaggcgtgggccaccgcacctggcc
aaatttttaatttttttctagagatagggtcttactgtgttgcccaggctggtgtcaaactcctgggctcaagcagatcctcctgcctcagcttcc
caaagtggtgggattataggtgtgagccactgcgcccagtcagtagccccctctttgcccctcactgagccctactggatgttcttggttgtgtg
acagtttccccatctattaaacagaaacccctatagcagaggggaggatgaggttggaaaatcaggagcattgttattctattcttgtgggatc
ggggaagcagacatctgggtggatgtttggggaatgctgggctcagttgaggaagtaggggggcccctggggcttacagggactggaa
gctctgagctggccagagggatgttgcaatcctgccagggtcttgtctatgctgtccctttcacaaccatccccctaccgccaggctgacacg
tggttgtgggggcacaaggccagccgaactagagtctgaggctgggctgaggacaccctccccatcagctgccagggtcactggcggtc
aaaggcagctggtggggaaggaattggactccagccctgggggacggatgtggtgatggtgggaagcaggcttggtgccaggagggg
catcagagggtgaataagagcagatagagtgtttgggggaggtagccagccaaagggggtgaggcccggtggaagggaagaagggg
catacactcagagctttgcagctgaaggttttaattttttgagatggggtctcactctgtctcaccaggctggagtgcagtggcgcaatcacag
ctcactgcagcccgggggatccggagagctcgtcgacggcgcgcc
GHpA
(SEQ ID No. 7)
Aattcctgcagcccgggtggcatccctgtgacccctccccagtgcctctcctggccctggaagttgccactccagtgcccaccagccttgtc
ctaataaaattaagttgcatcattttgtctgactaggtgtccttctataatattatggggtggaggggggtggtatggagcaaggggcaagttgg
gaagacaacctgtagggcctgcggggtctattgggaaccaagctggagtgcagtggcacaatcttggctcactgcaatctccgcctcctgg
gttcaagcgattctcctgcctcagcctcccgagttgttgggattccaggcatgcatgaccaggctcagctaatttttgtttttttggtagagacgg
ggtttcaccatattggccaggctggtctccaactcctaatctcaggtgatctacccaccttggcctcccaaattgctgggattacaggcgtgaa
ccactgctcccttccctgtccttctgattttaaaataactataccagcaggaggacgtccagacacagcataggctacctggccatgcccaac
cggtgggacatttgagttgcttgcttggcactgtcctctcatgcgttgggtccactcagtagatgcct
Construct
(SEQ ID No. 8)
AAACATCATCAATAATATACCTTATTTTGGATTGAAGCCAATATGATAATGAGGGGGTGGAGTTTGTG
ACGTGGCGCGGGGCGTGGGAACGGGGCGGGTGACGTAGTAGTGTGGCGGAAGTGTGATGTTGCAAGT
GTGGCGGAACACATGTAAGCGACGGATGTGGCAAAAGTGACGTTTTTGGTGTGCGCCGGTGTACACA
GGAAGTGACAATTTTCGCGCGGTTTTAGGCGGATGTTGTAGTAAATTTGGGCGTAACCGAGTAAGATT
TGGCCATTTTCGCGGGAAAACTGAATAAGAGGAAGTGAAATCTGAATAATTTTGTGTTACTCATAGCG
CGTAATATTTGTCTAGGGCCGCGGGGACTTTGACCGTTTACGTGGAGACTCGCCCAGGTGTTTTTCTCA
GGTGTTTTCCGCGTTCCGGGTCAAAGTTGGCGTTTTGATATCAAGCTTATCGATACCGTAAACAAGTCT
TTAATTCAAGCAAGACTTTAACAAGTTAAAAGGAGCTTATGGGTAGGAAGTAGTGTTATGATGTATGG
GCATAAAGGGTTTTAATGGGATAGTGAAAATGTCTATAATAATACTTAAATGGCTGCCCAATCACCTA
CAGGATTGATGTAAACATGGAAAAGGTCAAAAACTTGGGTCACTAAAATAGATGATTAATGGAGAGG
ATGAGGTTGATAGTTAAATGTAGATAAGTGGTCTTATTCTCAATAAAAATGTGAACATAAGGCGAGTT
TCTACAAAGATGGACAGGACTCATTCATGAAACAGCAAAAACTGGACATTTGTTCTAATCTTTGAAGA
GTATGAAAAATTCCTATTTTAAAGGTAAAACAGTAACTCACAGGAAATACCAACCCAACATAAAATCA
GAAACAATAGTCTAAAGTAATAAAAATCAAACGTTTGCACGATCAAATTATGAATGAAATTCACTACT
AAAATTCACACTGATTTTGTTTCATCCACAGTGTCAATGTTGTGATGCATTTCAATTGTGTGACACAGG
CAGACTGTGGATCAAAAGTGGTTTCTGGTGCGACTTACTCTCTTGAGTATACCTGCAGTCCCCTTTCTT
AAGTGTGTTAAAAAAAAAGGGGGATTTCTTCAATTCGCCAATACTCTAGCTCTCCATGTGCTTTCTAGG
AAACAAGTGTTAACCCACCTTATTTGTCAAACCTAGCTCCAAAGGACTTTTGACTCCCCACAAACCGA
TGTAGCTCAAGAGAGGGTATCTGTCACCAGTATGTATAGTGAAAAAAGTATCCCAAGTCCCAACAGCA
ATTCCTAAAAGGAGTTTATTTAAAAAACCACACACACCTGTAAAATAAGTATATATCCTCCAAGGTGA
CTAGTTTTAAAAAAACAGTATTGGCTTTGATGTAAAGTACTAGTGAATATGTTAGAAAAATCTCACTG
TAACCAAGTGAAATGAAAGCAAGTATGGTTTGCAGAGATTCAAAGAAAATATAAGAAAACCTACTGT
TGCCACTAAAAAGAATCATATATTAAATATACTCACACAATAGCTCTTCAGTCTGATAAAATCTACAG
TCATAGGAATGGATCTATCACTATTTCTATTCAGTGCTTTGATGTAATCCAGCAGGTCAGCAAAGAATT
TATAGCCCCCCTTGAGCACACAGAGGGCTACAATGTGATGGCCTCCCATCTCCTTCATCACATCTCGAG
CAAGACGTTCAGTCCTACAGAAATAAAATCAGGAATTTAATAGAAAGTTTCATACATTAAACTTTATA
ACAAACACCTCTTAGTCATTAAACTTCCACACCAACCTGGGCAATATAGTGAGACCCCATGCCTGCAA
AAAAAAAAAAATTAGCCAGGCATGGTAGCATGTACCTGTAGTCCCAGCTACTTGAGAGGTGAGGTGG
GAAAATCACTTTAGTGCAGGATGTTGAGGCTGGAGTGAACTGTGATTGTGCCACTGCACTCCAGCCTG
GACAATAGAGCAAGACCTTGTCTCAAAAAAATGCATTAAAAATTTTTTTTAAATCTTCCACGTATCAC
ATCCTTTGCCCTCATGTTTCATAAGGTAAAAAATTTGATACCTTCAAAAAAACCAAGCATACCACTATC
ATAATTTTTTTTAAATGCAAATAAAAACAAGATACCATTTTCACCTATCAGACTGGCAGGTTCTGATTA
AATGAAATTTTCTGGATAATATACAATATTAAGAGAGACTGTAGAAACTGGGCCAGTGGCTCATGCCT
GTAATCCCAGCACTTTGGGAGGCTGGGTAACATGGCGAACCCTGTTTCTACAAAATAAAAATATTAGC
TGGGAGTGGTGGCGCACACCTATAGTCCCAGCTACTCAGGAGGCTGAGGTGGAAGGATCGCTTGAAC
CCAGGAGGTTGAGACTGCAGTGAACTGTGATCATTCTGCTGCACTGCACCCCAGCCTGGGCAACAGAG
ACCTTGTCTCAAAAAAAAAAAAAAAAGAGACAAATTGTGAAGAGAAAGGTACTCTCATATAACATCA
GGAGTATAAAATGATTCAACTTCTTAGAGGAAAATTTGGCAATACCAAAATATTCAATAAACTCTTTC
CCCTTGACCCAGAAATTCCACTTGAATAAAGCTGAACAAGTACCAAACATGTAAAAGAATGTTTCTTC
TAGTACAGTCGGTAAGAACAAAATAGTGTCTATCAATAGTGGACTGGTTAAATCAGTTATGGTATCTC
CATAAGACAGAATGCTATGCAACCTTTAAAATATATTAGATAGCTCTAGACACACTAATATTAAAAGT
GTCCAATAACATTTAAAACTATACTCATACGTTAAAATATAAATGTATATATGTACTTTTGCATATAGT
ATACATGCATAGGCCAGTGCTTGAGAAGAAATGTGTACAGAAGGCTGAAAGGAGAGAACTTTAGTCT
TCTTGTTTATGGCCTCCATAGTTAGAATATTTTATAACACAAATATTTTGATATTATAATTTTAAAATAA
AAACACAGAATAGCCAGACATACAATGCAAGCATTCAATACCAGGTAAGGTTTTTCACTGTAATTGAC
TTAACAGAAAATTTTCAAGCTAGATGTGCATAATAATAAAAATCTGACCTTGCCTTCATGTGATTCAGC
CCCAGTCCATTACCCTGTTTAGGACTGAGAAATGCAAGACTCTGGCTAGAGTTCCTTCTTCCATCTCCC
TTCAATGTTTACTTTGTTCTGGTCCCTACAGAGTCCCACTATACCACAACTGATACTAAGTAATTAGTA
AGGCCCTCCTCTTTTATTTTTAATAAAGAAGATTTTAGAAAGCATCAGTTATTTAATAAGTTGGCCTAG
TTTATGTTCAAATAGCAAGTACTCAGAACAGCTGCTGATGTTTGAAATTAACACAAGAAAAAGTAAAA
AACCTCATTTTAAGATCTTACTTACCTGTCCATAATTAGTCCATGAGGAATAAACACCCTTTCCAAATC
CTCAGCATAATGATTAGGTATGCAAAATAAATCAAGGTCATAACCTGGTTCATCATCACTAATCTGAA
AAAGAAATATAGCTGTTTCAATGAGAGCATTACAGGATACAAACATTTGATTGGATTAAGATGTTAAA
AAATAACCTTAGTCTATCAGAGAAATTTAGGTGTAAGATGATATTAGTAACTGTTAACTTTGTAGGTAT
GATAATGAATTATGTAAGAAAACAACAGGCCGGGCGGGTTGGTTCACACGTGTAATCCCAGCACTTTG
GGAGGCTGAGGCAGGCAGACTGCCTGAGCTCAGGAGTTCGAGACCAGCCTGGGCAACACGGTGAAAT
CCCGTCTCTACTAAAAATACAAAAAAATTAGCCGGGTGTGGTGACACATGCCTGTAGTCCCAGCTACT
TGGGAGGCTGAGGCAGGAGAATCACTTGAACCTGGGAGGTGAAGGTTGCAGTGAGCCAAGATGGCAC
CACTTCACTCCAGCCTGGGAAACAGAGCAAGACTCTGTCTCTGAGCTGAGATGGCACCACTTCACTCC
AGCCTGGGAAACAGAGCAAGACTCTGTCTCAAAAAAAACAAAACACACAAACAAAAAAACAGGCTG
GGCGCGGTGGCTCACGCCTGTAATCCCAGCACTTTGGGAGGCCGAGGCGGGTGGATCACCTGAGGTCA
GGAGTTCCAGACCAGCCTTGTCAACATGGTGAAACCTCCCCCCGCCGTCTCTACTAAAAATACAAAAA
TTAGCCAGGCGTGGTGGCAGGAGCCTGTAATCCCAGCTACTTGGGAGGCTGAGGCAGGAGAATCGCTT
GTACCCAGAAGGCAGAGGTTGCACTGAGCTGAGATGGCACCATTGCACTCCAGCCTGGGGGACAAGA
GCGAGATTTCGTCTTTAAAAAACAAAAACAAAACAAAAAACCATGTAACTATATGTCTTAGTCATCTT
AGTCAAGAATGTAGAAGTAAAGTGATAAGATATGGAATTTCCTTTAGGTCACAAAGAGAAAAAGAAA
AATTTTAAAGAGCTAAGACAAACGCAGCAAAATCTTTATATTTAATAATATTCTAAACATGGGTGATG
AACATACGGGTATTCATTATACTATTCTCTCCACTTTTGAGTATGTTTGAAAATTTAGTAAAACAAGTT
TTAACACACTGTAGTCTAACAAGATAAAATATCACACTGAACAGGAAAAACTGGCATGGTGTGGTGG
CTCACACTTGTAATCCCAGTGCTTTGGGAGGCTGAGACAGGAGAGTTGCTTGAGGCCAGGAGTTCAAG
ACCGACATGGGGAATGTAGCAAGACCCCGTCCCTACAAAAAACTTTGTAAAAATTTGCCAGGTATGGT
GGTGCATACCTGTAGTCCCAGCTACTCGGGAGGCGGAGGCAGAAGGAATCACTTGAGCCCAGGAGTT
TGAGGCTGCAGTGAGCTACGATCATACCACAGCACTCCAGCGTGGACAACAGAGTAAGACCCTATCTC
AAAAACAAAACAAAACAAAACAAACAAAAAAAACCACAAGAAAAACTGCTGGCTGATGCAGCGGCT
CATGCCTGTAATCCCAGTATTTTGGGAGGCCCAGGTGGGCGTATCACCTGAGGTCAGGAGTTAGAGAC
CAGCCTGGCCAACATGGTGAAACCCCATCTCTACTAAAAATACAAAATTAGCCAGGCATGTGGCACGC
GCCTGTAGTCCCAGTTACTGGGAGGCTGAAGCAGGAGGATCACCTGAGCCCGGGAGGTGGAGGTTGC
AGTGAGCCGAGATCACACCACTGCACTCCAGCCTGGGTGACACAGCAATACCCTACCTCAAAATAAA
AAAGAAAAAGAAAAGAAAAGTTGCTGTCCCCGCTACCCCAATCCCAAATCCAAACAGCCTCTCTCATC
TCACAGTAAGGGGGAAAAATCACCCAAAAAAGCTAAGTGATCTTTTGAAAACCCAAACTCTTAGAAG
TCTAAGATTATTATAGTCAACTCATGAAGTGTCATCATAAAAGATACTCTAATATTATTTAAGTAGAAC
CACATATTGGTTGTCTTGGTATGTCTAGCCCCTGGCATACAAAATATTTAATAACACTGATATGGTACC
TGTGATGTGAAAATGTACTATGAGTACAGCTTTATAAATACTATATATGTACCTATATACAGAAAAAA
ATACAACAAAATCATAAAAGCACTTATCTTTGAAAGAGGAGTTACAGCAATTTTATTTAGTTCTTTATT
GCTTTGCTATATATTCTAAATTTTTTTCAATGAATATATATCACTTTTAAAAAAATTCAATGGTCTTTCT
TATAAATTATCTTTGGCAGCATGCGTTTTTATATATACATATAAAATGTATGGGAAATTTTTAAAGGAT
ACATTAAATTAAAGCAAAATATACAAACAAAAAATCAGAATACAAAAAGATAAAAAGATTGGGAAG
GGAGGGAGGGAGTAAGGAGGAAGGGTGGGTGGGTATAGAGAAATATACCAAATAATGGTAAGAAGT
GGGGTCTTGACACTTTCTACACTTTTTTTAAATAAAAAAAATTTTTTTCTCTCTCTTTTTTTTTTTTAGAG
ACGAAGTCTCGCTATGTTGCCCAGGCTGGTCTTGAACTCCTGGGATCAAGAGATCCTCCTGCCTCAGCC
TCCCAAGGTGCTTGGATTACAGGTGTGAGCCACCACGCCTGGTCACTTTCTACACTTTAATATATATAT
TTTTTCATTTTCAATGTCATTTTTATTAGTTAATTTATAATACCCATTCACCATTATATTCAAAGTCTATT
TGAAGAAATAAACCAGAAAGAATGAAATACTCTAGCTCACATGCTATTCAATACTAAATTACCTTTCA
AATCACATTCAAGAAGCTGATGATTTAAGCTTTGGCGGTTTCCAATAAATATTGGTCAAACCATAATT
AAATCTCAATATATCAGTTAGTACCTATTGAGCATCTCCTTTTACAACCTAAGCATTGTATTAGGTGCT
TAAATACAAGCAGCTTGACTTTTAATACATTTAAAAATACATATTTAAGACTTAAAATCTTATTTATGG
AATTCAGTTATATTTTGAGGTTTCCAGTGCTGAGAAATTTGAGGTTTGTGCTGTCTTTCAGTCCCCAAA
GCTCAGTTCTGAGTTCTCAGACTTTGGTGGAACTTCATGTATTGTCAGGTTGGCCCGTAATACCTGTGG
GACAACTTCAGCCCCTGTGCACATGGCCAGGAGGCTGGTTGCAAACATTTTCAGGTAGGTGGACCAGG
ACATGCCCCTGGTCATGGCCAGGTGGAGGCATAGTGCTATACAGCAGGCAGAAGTCAATATTGATTTG
TTTTTAAAGAAACATGTACTACTTTCATAAGCAGAAAAAATTTCTATTCTTGGGGGAAAAGATTATGC
CAGATCCTCTAGGATTAAATGCTGATGCATCTGCTAAACCTTCACATATCAGAACATATTTACTATAGA
AAGAATGAAAATGGGACATTTGTGTGTCACCTATGTGAACATTCCAAAAATATTTTACAACAACTAAG
TATTTTATAAATTTTATGAACTGAAATTTAGTTCAAGTTCTAGGAAAATACAAACCTTGCTAGATATTA
TAAAAATGATACAATATATATTCATTTCAGGCTCATCAGAATATATCTGTTATCACTTGACAAGAATGA
AAATGCACCATTTTGTAGTGCTTTAAAATCAGGAAGATCCAGAGTACTAAAAATGACTTCTTCCTTGA
AGCTTACTCACCAACTTCCTCCCAGTTACTCACTGCTTCTGCCACAAGCATAAACTAGGACCCAGCCAG
AACTCCCTTGAAATATACACTTGCAACGATTACTGCATCTATCAAAATGGTTCAGTGCCTGGCTACAG
GTTCTGCAGATCGACTAAGAATTTGAAAAGTCTTGTTTATTTCAAAGGAAGCCCATGTGAATTCTGCCC
AGAGTTCATCCCAGATATGCAGTCTAAGAATACAGACAGATCAGCAGAGATGTATTCTAAAACAGGA
ATTCTGGCAATATAACAAATTGATTTCCAATCAAAACAGATTTACATACCATACTTATGTCAAGAAGTT
GTTTTGTTTTATTGCATCCTAGATTTTATTTTTTTGATTTATGGTTTACTTTAAGCATAAAAAATTTGTCA
ATACAACTCTTCCCAAAAGGCATAAACAAAAATTCATAAAACTTGCATCACTTGAGATACTTCAGGTA
TGAATTCACAACTTTGTTACAACTTACTATATATATGCACACATATATATATATTTGGGTATATTGGGG
GGGTTCTAATTTAAGAAATGCATAATTGGCTATAGACAGACAGTTGTCTGGAATGAAAATCAATACTT
TTGCTATAATCGATTACTGAAATAATTTTACTTTCCAGTAAAACTGGCATTATAATTTTTTTTAATTTTT
AAAACTTCATAATTTTTTGCCAGACTGACCCATGTAAACATACAAATTACTAATAATTATGCACGTCAC
ATCTGTAATAATGGCCTTCATGTAAACATTTTTGTGGTTTACACATAAAATCTCTAATTACAAAGCTAT
ATTATCTAAAATTACAGTAAGCAAGAAAATTAATCCAAGCTAAGACAATACTTGCAACATCAATTCAT
CATCTGTGACAAGGACTGCTTAAGTCTCTTTGTGGTTAAAAAGGAAAAAAAAAAAAAAGACATGTTG
GCCAGATGCGGTGGCTCACACCTGTAATCCCAGCACTTTGGGAGGCTGAGGTGGGCGGATCACCCCTG
GCCTGCCCAACATGGTGAAACCCCGTCTCTACTAAAAACACAAAAATTAGCTGGGCGTGGTGGCGGGC
GCCTGTAATTCCAGCTACTCGGGAGGCTGAGGCAGGAGAATTGCTAGAACCCAGGAGGCAGAGATTG
CAGTGAGCTGAGATTGCACCATTGCACTACAGTCTGGGCAACAAAAGTGAAACTCCATCTTAAAAAAA
AAAAGACAATGTTCGTGGGTCCAAACAAGACTTAATGGAAGTGAGTCTAAAAATGAGCTATGTGGGC
CAGGCGTAGTGGCTCCCACCTGTAATCCCAGCACTTTGGGAGGCCGAAGCAGGCAGATCATGAGGTCA
GGAGATGGAGACCATCCTGGCCAACACGGTGAAATCCTGTCTCTACAAAAATTAGCTGGGCGTGGTGG
TGCCTGCCTGTAATCCCAGCTACTCAGAAGGCTCAGGCAGGAGAATCGCTTGAACCAGGGAGTCGGTG
GCTAGAGTGAGCCGAGATTTGCATCACTGCACTCCTGCCTGGTGACAGAGCAAGACTCCATCTCAAAA
AAAACAAACAAAAATAAAAGATAAAAATGAGCTATGTGAATTAAAAGAGGTATAACAATAGATAAA
CCATATTTTATTTAATTCCTAGTAATGAGTAATATTTCCAAACTTCTGGAATGGGCAGAAATTGCTAGT
TGGCATATTTTTACCTTTTATATTCAGATACATTAAAATTCTCAAAAAAAAACACCTCAAAGCAGATGA
TCCGCCATCTCCTTGGATAATTTGTGTTAACTCAGGATAACAGAAAACCAAAATTATGAGTTACTGAT
GCAATATTCCTAAATGTAAAAATAATTAAAGCTAATAGTAGATTCATCTTCCAATTTCATATCAGTCTT
ACAAATAAACTACATATATAACTTGCTTGCCTTCCCTTCTGAGGGATAAAGCTGTTAGAAGAATTAAA
ATCAGCATTCTTGACTATTCAACCAAGGGAGGGATAAATTATTACTCATTCTAGGGACATGGGCTCAT
AACTACTACATGTGTAAGGACATGAATTTACCCAATATTACAATTTTTCCTTTTATTAGTGTGTACAGT
GGAAGAATAGACATGTTCACTCTGGACAAAAAAAAAATTATACTTATCAGTTATCAGAAGCACAATGC
TGAAGACAGTAGTTCCATAACAATTTGAAGTATGTGATCGAACTAGTAGATTATCTTAGTAGTAGTGA
ATTATTGTAAATGTTAGTAATTTGGCAGCCACTGGGCAGAAAAATAAGAATTGAGGCTCAATATTGAT
ATTAATGGTGGTGATTGACACATAAATTTTATCAAGTCTACACAATATAAAATTACAGAAAGGTAGAA
GAGTATACCAGTACAACTTCAACATATCTTCACTACAAGGGAGTAAAATGACATGGCCTAGTTACTAT
CTAATGAACTGCAGAAAACTAAAAGAAAACTCCAAGGCAACTCTTCTCTGCTGATCTGGTTGGTCCTT
TTCCTACCTTTTGCAATACCCAGATACAAACAATGGATAGAAAACAAAGTAGACTTGTAGTATGCAGG
TCACAGTGCTAAATTCACAGAAAGAAACCCCTGAACTGAACTGCTCTATTTCCTGGTGGTCACAAAGA
GTAATTCTGGTTTACACCTACAGATTGATGTCAATCTACACCCTGTTGATAACAGTGTGGCCAAGGAC
AAAAAAAAGGTGCTCCGTTTTACCAATTCTGTAAAAAATTATTGGCAGGGTAAGCTCGGCTAGGGCAG
GATTACATTTCTAGGACTACCATCCCCGAAATTTAGAAGATATTATATCCACATAAAGCATATCTTTCA
CATTAATTTGCAAAAATCTAAAAGCTTTTTCTTAGCTCAAGTGTGTCCAAGTTTACCCTGGCAGTTTAA
AACGATAGTTACAAGCAGCATGGGTTGTATCAGACACATTTGAGGGCCAATTTCATGTAAGTGATATT
GGGCAAGTTACTTCAACTATCTGTGCCTCCAAGGTCATACTAGTGTTTATTTACCTAAAGGGTACCTGT
TATGTAACTTTAGGGTGTTTACATTAGATAATGCCTGCAAAATATTTACTTCAACGCCTAAAACATAGT
TAAGTATTCAATAAATACCTACTATTGTCACTACTAACTTAAAAGTTTAGAGATTAAGAGCAGAATCT
GGGGTGAGACAAACTTAGGTTCAAATCCTAGTATTGTTGGGTAATCTTGGGCAAGTTACTTAACCTCTC
TGATTTGTGTAATTTAAAAAATTAGTTAATATACATAACAGGGCTTAGAAGAGTATCTAGCACATAGC
ACCATTTAAGCATTTGTTATTGCTAACATGCAAACAATTTAAGGGAAAGAAATTTTTTAAAAAGGAAG
AGGGATTTGCAAACTAAAAACAATGAGTATCTTATGTTCAAAGAAAACTAACAAACAGCCAGCTCTA
GCAATAATTAAATTCACTATATACTGGGGCAGGCATCACACCCCAAAGCTAAAAGCGTCTACCTAGGC
CAGGCACGGTGGCTCATGCCTGTAATCCCAGCACTTTGGGAAGCAGAGGCGGGCAGATCGCTTGAGCT
CAGGAGTTCAAGACCAGCCTGGACAACATGGCAAAACACCATCTCTACAAAAAATACAAATATTAGG
CCGGGCGCAGTGGCTCACGCCTGTAATCCCAGCACTTTGGGAGGCCAAGGCGGGTGGATCACCTGAG
ATCAGGAGTTCGAGAGTAGCCTGGCCAACATGGTGAAACCTCGTCTCTATTAAAAATACAAAAAATTA
GCCAGGCATGGTGGCAGGCGCCTGTAATCCCAGCTACTCAGGGGGATGAGGTAGGAGAATCGCTTGA
ACCCGGGAGGCAGAGGTTGCACTGAGCCGAGATCATGCCACTGTACTCCAGCCCGGGCAACAAGAGC
GAAACTCCATCTCAAAAAATAAATAAATAAATAAATAAAATAAAGTACAAATATTAGCCAGGGATGG
TGGTGCGCACCTGTAGTCCCAGCTACTTGGGAGGCTGAAGTGGGAGAATCCCCTGAGCCTGGGGAGA
ATCACCCGAGCCCGGGAAGTCGAGGCTGCAGTGAGCAGTGATTGTGCCACTGCACTCCATCCTAGGTG
ACAGAGTGAGACCCTGTCTCAAAAAAAAGAAATTGGCAGAATTAAGTAAGTTGATGTTTAGAGATGA
AAAATCAACATTTTTTCCTCAGCAACTGAATAAAAACAACAGCCACTACCATTTTTTTGAGTACCTATT
TGTAGCCTATTTTTTAACTGGTATTACTCGAGAGAGAGAGAGCTAGGTTCGAGACAGAGCTCCTTCTCT
TAATAACTGTATGACCTAGGGTATGTCTGTTAGCCTCTCTGAGGCTTCAAAGGTTCCTCATCTGTAAAA
TGGTAATAATCATACCATTGCTACAGGGCTGTTTTGAAGACTAATTAGGACTATGTAAGTAAACATGA
TGATGGCTATTATTACTGTTCCCCGCCAGGGGCCATGCAAGGGTTGCTGATTCACATAGACTGTCTTAT
AATCCTCTCAATAACTCCAAGAGGTAGCCAGCACCTCAGATATACATAAAATGACTTAAGCCCAGAGA
GGTGAAGTAAGTTGCCCACAGCCACACAACTAGTAAATAGCCCAAACAAGCTGGATTCCCAGTTAGA
CTCCGTTAATAGCACTGCTCTTTACCTTAAGTCATTACAATGCCTAATATGAAATAGAATCGCTTCTTT
CTTAGGGTTCAAGTGGTTAATTATTTAATGTATTCATTCAACAAACCATCATCGAGGACCTCTTACAAG
CCAAGTACTGTGCTAAGTGCTAGAGTTACGGCGGTGATTCCTGCCCTTAAAAAGTTTTAGTGGGAGAA
ACAACAGGTAACCAGGTCATTGCCAAAACAACAAAAATAATCATAATAAAGCAGGCTAAAGCATATT
TAACTGGCCGGGGTTTTGACTATTTTAGCAAGCATGATCAGAACGGTTGAGGAGGGAGGCCAGCAGCT
TGGCCGGTTCAACAAACAAGAAAAAACCAGTGAGGGTGGAGCTAAGATACCAGAGGCTGATTACGGT
TAAGAATGTTCTTGAAGGTAAGGACCAGATTCTCATTTTCTATATCCTGGGGCATCGGTCAGCATGGA
ATCTGGATTCTAGCACATGTGAATTTCGGCTTGAAATGACCTAATGCCTTTTCCCTAGTTCCTTCGTGT
GTCAAATACGCATGGTTACCGCTACCAGAGCTGTAGTGGGGCTTCAATGAGGCCATGAGCATCTCCAT
AAAGATGAACTACAGTGTGTGCAAAACTAAAGGCAAAACCTGGTCCCCACACGCCCTCCCAGGTGGT
CGCTTTCCGTGCCGAGGCCCCTCCAGAGGTGCCCCGAGAACCTCACCATCGCACCCCAAACTTCCAGG
GAAGGGCCTCTCCCGAGAAAGCCCCCACGCCCCCACCCCGCGCCATCATTCCCGAATCTGCCCTCGGC
CCCTCCCCGCAGCACGCTCGCAGGCGGCACATGTCAACCAAAACGCCATTTCCACCTTCTCTTCCCACA
CGCAGTCCTCTTTTCCCAGGGCTCCCCCGAGGAGGGACCCACCCCAAACCCCGCCATTCCGTCCTCCCT
GCCGCCCTCGCGTGACGTAAAGCCGAACCCGGGAAACTGGCCGCCCCCGCCTGCGGGGTTCCCTGGGC
CCGGCCGCTCTAGAACTAGTGGATCCCAATTGAAGGCCTGGTCTAAATGACTCCAAAATCACCACTTA
ATTCAAGAGACTGATTTCCCTGAGTCAGGCCCCTTAAAGCAGCTATTTCAATGGGACAGGGAAACAAC
CCTAGGATCTGGATTAGAATCACTTGGGGGCTGCCACACCCCCAGGGCTCTGATCCTGCCCTTCTCCCA
CACGCACATTCACATACTGCTGCAGTGACCTTCCATTTCTAATGGGTTCCTGGGCCATCTGTCAGGTAT
AGGGAATGGAAAAGGGGTTGGGGAGGCTCTGCTTCAGAAAGTTTGTGTCAGGGGCTCCCAGAGCCTC
CACAGATAGATAGCAGGGGTCCCCACCCTACCATGGCAGCTATAAATGTGATCAACATTTATTGGCCT
AGGATACAGCAGTTAGCAAAATGCCTGATGTAGTTCCCACTCCGTGGAGGTTGCAGGCTAGCTCTTTC
CTAATGAGCTTTACAGCAGAAGCTGTTTTATCGTTAAGTGCCCCACAGAGACACTTTACCAGGAGGCT
GGGAGAGTTCTCCAGATTTGGGAGAGGCGCAGAGACAGTGTGTGAGCCGAGCCCTGTCTCAGCAATC
CACCTGGAGGAGCTAGAGTATCCTCCTCCCTTTACCATTCAGACCGAGAGAAAAAGCCCAGCTTGTGT
GCACCCTCGTGGGGTTAAGGCGAGCTGTTCCTGGTTTAAAGCCTTTCAGTATTTGTTTTGATGTAAGGC
TCTGTGGTTTGGGGGGGAACATCTGTAAACATTATTAGTTGATTTGGGGTTTGTCTTTGATGGTTTCTAT
CTGCAATTATCGTCATGTATATTTAAGTGTCTGTTATAGAAAACCCACACCCACTGTCCTGTAAACTTT
TCTCAGTGTCCAGACTTTCTGTAATCACATTTTAATTGCCACCTCGTATTTCACCTCTACATTTGAAATC
TGGCGTCTGTTTCAAGCCAGTGTGTTTTTTCTTCGTTCTGTAATAAACAGCCAGGAGAAAAGTGCCTCT
ATGTTTTTATTTTTCAAGGGAGTATTCAGTACCTACAAACCCAAGTCAGGAAGCCTGCTAGTGGCTTTG
GTTCTTTCAGAGGCTGCTCGATGCCTTGTGTGTCAGAAAGAAAGATTCAGCAGTTTTGCATCATGGCA
AAGAAGCCTGTTATTTTGGGGCTCAGCCCCTCATTTTATAGAGGATGAAACAGAGGGGGATGGGAGGT
CACAAAGACAACTGCCCCGGGAGCAGGTGTGGGGGAGACTTGCCCTGAGGGTCTAGACGCTCTGCAC
CACCGTCCTGTCTCCCTTGCTGAAGACCACACATGCCCTTCTTTGACCAGACCCTGCCACCTGATAGGC
CAGGACCTGGTAGGCGGGTACCCAGGTTTCATGGATGGAACCACATCTCCCCAAAAGTGGGGAGGTA
GCTACTGGGATGCACGCCTCCCGCCATGTGCTATAGGAGAGCAGCTGAAGCAACAGTTGGGATCAGAT
GTAGTCACAATTGAATGCATCATCACATTTATCCCTCTAAGTGGCTGGGAGAGTTGATATCCTCATCCC
TAAGGTACAAAATGTTCCAATTTGATCAGTGGCTTTCAGGAGCTGAGAAAGGCATGTGCTCTGAGGCA
GAGCTGTTATGTCCCGCAGAGCCTAAAAATGCTCTAAGAACATGCTCCCTGCCAAAATTCTCAATGGC
TGTGACAAGGGACAACGATCGACCAATGGGGGTGGAAGCAGACCTCCGCAGTCCAGGGGCCAGAGCT
AGGACAGAGGGGTCGGAGAAAGAGTCATTTTCCCAACACTCCAGCTCTTGGCCAGTCCTCACACAGTC
CCCTCCTGCTTCCTGCTGAGAGAGATATCCTCATAGGTCTGGGTAAAGTCCTTCAGTCAGCTTTCATTC
CCTGTCACCAACTTTGTCTCTGTTCTCCCTGCCCGTCTCAGGCAGCACTCCTCAGGAAACCTCTCCAAG
AGCCAGCCTCACTGCAGCGCCCACTATTGTCCCTCTGCCTCAAGTGTCCCATCCATGCCAGGCCCCAGG
CAGGCTGCAGCTTTCCCTCAGGGCCACACCAAAGCACTTGGGCTCAGCTGTGCTGTCCCCCTCCATCAC
TGAGCTCAGGGGCAGCAGGGGTGGGGTGCCAGGAGGCCCATTCACCCTTCTCTGGCTCTGTGTTGGAC
CCACCTGCCCAGCCACTGCTGCTTAGAACCTACCCGCTGGGAAAATGAAGCCCTCCCGGAGGGGCCAC
CTCAACCTGAGAGCCTCACGGATCACAGTTGTCCCCACTCAGCTCTGCCAGCCCTCAGAGACCCATAG
ATAAAAGCTGAGCTTGGCTCGCAGAGCTGGTTCCATCTTCCATTCCCAGAGGGTTCAACTTCCTACCCC
AACCACACAGGGAACCTCAAGGCTGAGCCAGTGTGGGCTGCAGTGCAGACCAGCTTCCTGGACACGT
CCTGCCACCTGACCCCAGGCTGGCCTCACTGCCCCTGGCACTCCTGACCCTATCCTCATTCCTCCTGGC
AGTGCGTGTTCTGCCATTCCGCTTTCCCTTAGCTGTCCTCTCACTGTACTGTCAGCTTCTCCTTTTCCAG
GTGCCCCCCAGGGGCTTTCCACATGACCCTGTCACCCCACAGCCCATCCAGCACCAATTCCAGCTCTCT
GCCACCCTTCAAAGGAGTGACAGTGCCCTGCTTCACCTCCCACTCACCCCTCAACCCAGAGCAATCTG
GCTCCAGTCTTGCCTCCTTCCCCCTAAGTACTCTAGTCACAGTTCCAAATTCCTCCTGGTCATAAAGCC
AAATGAAGCTTCCTGGTCCTCAGCGGACTTGCCACTTCAGCAGTACTGGACTCTCTCCTCCCAGAAACC
TGTTTCCCCTTGGCTCCTGGAGCCCACACTCTGCTGGAATCCTTCTGCCTCTCTGGCCTGTAGCCTGGCC
CTCTCTCCCAACCTGAGGTCCATTCTCTCCTGCTCCTCCACAAGATGTTGCTCCTTCCATTACTTCCTCC
CTCTCAACCAAAGCTCCTTCATTAGCTCTTTATCTTCTGGTTTCTTCCCCTGGGCAGACGAATGGATTCA
AGAGCCTGTGGCCCAGCAGCCCAGCACTCCAGGATCTCAGCACTTCAGCATCCCAGTACCCTAGCATC
TCAATACCCCAGCACCCCAGCACCATAGTATTCCAGCACCCCATTGTCCAAGCATCTCAGCACTCCAG
CATCCCAGCACCCCAACACTCCAGCAGCCCAGAATCTCAGCACCCTAGCACTGCAGCATCTCAGGACC
CCAGCACTTCAGCATCCCAGCACACTAGTACTCCAGCATCTCGGCACCCCAGCACCTAGGCATCCCAA
CACCCAGCACCCCAGCACTTAAGCATCCCACCACTACAGTATCTCAACACTCCAGCACCCCAGCACCA
TAGTGTTCCAGCACCCCAGCATCCCAACACCCCAGCACTTAAGCATCCCAACACCTCGGCATCCCAAC
ACCCCAGCACTGCAGCATCTCAGCACCTTAGCATCCCAGTGCCCTAGCATCTCAATGCTCCAGCACAC
CAGTACTACAGTATTCCAGCACCCCAGCACTCCAGCATCTCAGCACTGCAGCACTGCAGCACTCCAGC
ATCCCAAAATCCCAGCATCCCAACACCCCAGCAGACCAGCAGACCAGCATCTCAGCACCGCAGCATCC
AAGGACTATCCCAGCATCCCAGCAACCCAGCACCTCAGCATCCCAACACCCCAGCATTTCAGCATGGC
AACACCCCAGTACCCCAGCACTTCAGCACCCCAGTATCCCAGCATCTCAGCGACCCAGTATCACAAAA
CCTCAGCATCCTAGCACCCCAGCACCCCAGCACCTTAGCACCTTAGCATCCCAGCATCTCAGCGCCTC
AGCATCTTGATATTCTGGCTGAGGTCAGCGTGGTGTATCTAGTCAGGGTCCTAACTTTCACTTCGCAGG
GAAATGCTGCTGGACTGGGTCTCATGTTGGGCTGAAGCTCTCTAGACCCCTTGAAGACAGCATAAAAG
AGCTTGGAGACGCTGGGTGTCCCCCATGGAAGAGTTCACTCTCATCCTGCTTTGACAACAGCCTTCTCT
GGGGTCCCTCACGGGCCCCTCTTTCTTACTGCAAGTTTGTCTCTGAGAAGACTGTGATGCAGAAGTCAC
TCAGCTGCCTGTGGCTCCTGAAGAGCTGAAGGTGGAGGCCTGTAGGCCTCCCTATGAGAGGCGCAGAA
AAAACCATGATTGCTAGTGGGGAGGTGCTCCCTCTACAACCCACTCCATAATCTGCCCCCGCCCAGCT
CTGAGGCCAGCCCCAGGGGAAAATGCCAGATCCCCAGGGAGGTGTGTGAGACCTCAGGGGCTCCCTC
CTCCCTTACAGCAGGCTCAGGCCCCTGGGGGCCTCAGGGCCAAGGTCTGTGGGTAAGCTACTATCTCT
CACTTGTCCTCTAGCCACAAAAGCCAGGGAGATCTGGCAATGGACATGAGGTTCTGAAGAAGCACAT
ATGACTGGCTTCCTAATGCGTGGTTGTTCAGTGATTCAATAAACACGCATGGGCCAGGCATGGGGAAA
TAGACAAACATGATCCCCAACCTCTCCCAGAGTGAACTGGGAGGGAGGAGTGTTCATCCCTCAGGATT
ACACCAGAGAAACAAACCAGCAGGAGATATATATGGTTTTGGGGGGTCAAGAAAGAGGAAAAACCTG
GCAAGGCAAGTCCAAAATCATAGGACAGGCTGTCAGGAAGGGCAGCCTGGAACCTCTCAAGCAGGAG
CTGATGCTGCAGTCCACAGGCAGAATTTCTTCTTCCTCGGGGAAATCTCAGCTTTGTTCTTAAGGCCTT
TCAACTGATTGGCTGAGGTCTGCCCCTTCCCCCACATTCTCCAGGATAATCTTCCTTACTTAAAGTCAA
CTATTAATCACAGCTACAAAATCCCTTCACAGCTACACATAGATCAGTGTTTGATTGACGAACAGCCC
CTACAGCCTAGCCAAGTTGACACATAAAACTAACCATCACAGGGGGACAAATGATGTAAACACATCA
ACAAATAAAACAGTAACAAGTTAAGGTCTATGGAAAAAACACAGAAGGGGCAGAGAGAAAGAAAGC
AAGAAGGAGAGTCCCAGTTTGCTAGGGCTTGTGGGAAGTGGGGAGCAGTTCTCTTTAGCTAGGATATT
TGGGAAAGGCATATCTGAAGGAGTGATATTTGAGCTTAGATTAAAAGATGGGAAGGAGCAAGCCATG
CAAAGAGCTAGGATGTTCCAAGCAGAGACGGAACAGCAAGTGCAAATGTCAGGAGGAATAGAAGGA
GGCTGGTGGGTGGGGTCCAGTGAGCAAGAGGAGGGCAGGCAGGAGAGGGGATGGGGAGGTGGGCAG
GCCCAGACCACCCAGGGCCCTGGAGACTATCCTGATCCAACAAGGGAAGCCTTGAGTCACTTCAGTGT
CCATGTGGAGAATGGACCTCAGACTGAATGAGGGAGGCAGTAAGGAGGGCCTCTACCTCCAGGGCTT
CGCCCTGTGGACTGCGCATAGACATCTCCAACTCAGAAAGTCTGAACCAAACTTTCCATAGTTCCCCC
AAGTCTGGGCATCCTCCTACTCAGTGAAAGGCAGCCATCACACCTCCCTGCCCTGCTCCCGGATGCCC
CAAATCCTCTTGGTCTCCAAGTCCAGAACCTGAGACTTGTCCTTGATGTTTGTCTTTCCCTCACCCTTTC
TGTATTCTGGGAAGATGGGTTTTTTTCCCCCAGATGAATCTGTAAAACTTCTGTGATCACAATAAAAAT
TCTGGCAGTATTATTTTCTGGAACATGACAAAGTGATTCAAAATTATTTATCTGGAAGACTACAAAAC
AAGAATAGCCAGGAAATTTCTAAAAAGAAAGAAGAAGGAGGAGGAGAAAGAAGGAGGAGGAAAAG
GAGGAGAAGAAGAAAAGAAAAAGAACCAAGAAAGGGTTCTAGCTCTACCAAATATTAAAACATATC
ATGAAGCTATTTAAAACAATATGGTTGTGGATACTGAAAAAGATGTGAATAAAGTGGAAGGAAAATA
AATAGAAATGCACATGGGGATTGAGACTGTGAAAAAGGCAGCATCTCACATCAGTGAGGGATGTTCA
ACACCTGGTGTTGGGAAAACTGGCTAGTCATTTAAACCAAACAACTGGGTCCTCTACCTCACTCCTGA
CATTAAGATACATTTAGATGATTCAAAGAGTAAGACAGAAAAAATAACACGTGAAAACACTATCAGA
AAACAACGTGGGCCAGGTGTGGTGGGTCACGCCTGTAATCCCAGCACTTTGGGAGGCCGAGGCAGAC
AGATCACCTGAGGTGGGGAGTTCAAGACCAGCCTGACCAACATGGTGAAATCCTGTCTCTACTAAAAA
TACAAAATTAGCTGAGCGTGGTGGCGCATGCCTGTAATCCCAGCTACTCAGGAGGCCGAGGCAGGAG
AATCACTTGAACCTGGGAGGCAGAGGTTGTGGTGAGCCGAGATCACGCCATTGCACTCCAGCCTGGGC
AACAAGAGTGAAAATCCATCTAAAAAAAAAAAAAAAAGCCAAGGTGGATATTTTTATAGTATCAGGG
TAGATCAAGCTTCTCCAATCATGACATGAAACCCAGAAACCATAAAAGAAAAGAATGATAAAATTGC
CCACGTAAAGTAAAAAGCTTGCACACAGAAAAACACCATACAGGTTACAAGATGAGCAGCAAAATCA
GAGAAAAAACATTGCAATTCAGGACACACAGAGGCTATTGTTCCTAATATTTAAAAATAAAAGTAGTG
GATTGTCTACAAAAAGATGAAGACAAGAATTTCAGAAAACCAAATACTGCATGTTTTCACTTACAAGT
GGAAGCTAAACACTGAGTACACGTGTACACAAAGAATGGAACCATAGGCCAGGCACCGTGGCTCACG
CCTGTAATCCCAGTACTTTGCGAGGCCGAAGCGGGCGGATCACCTGAGGTGAGGAGTTCGAGACCATC
CTGGCCAACATGGTGAAACCCAGTCTCTACTAAAAATACAAAAATTAGCCGGGCGTGGTGGTGGGTGC
CTGTAATCCCAGCTACTCGGGAGGCTGCGGCAGTAGAATCGCTTGAACCCTGGAGGTGGACCTTGCAG
TGAGCCGAGATCGCACCACTGCACTCCAGCCTGGGCAACAGAGTGAGACTCCATCTCAAAAAAAAAA
AAAAGGAATAGAACAATAGACACTGGGGCCTACTTGAGGGAGGAGGGTGAGGATCAAAAACCTGCCT
ATCAGGTACTATGCTTATTACCTGGGTGGTGAAATAATCTGTACACCAAACCCCAGTGACATGCAATT
TACCGATGTAACAAACCTGCCCATGTACCCGCTGAACCTAAAATAAAAGTTGGAAAAAAATATAGAA
ATTTTCTTTGTAATAGCCAAAAACTGCAAACAGCCCAGGTGTCTATTAGTAGAATGCATAAACAAACT
CGGGCATGTTCATACAATGTAAAACTACTCATCAATAAAAAGTGATACTTCTCAGCAATGAAAAGAAA
CTAGCTACTGATACCAGCTACAACATGGATGGATTTCAAGTGCTTTATGATGAGAGCAAGAAGCCAGA
CACAAAAGTGTCTATATATATATACAGTATATATACGTATATATACACATATATACAGTATATATATAC
ATATACATGTATATATATACTGTATATATACTGTATATATATACACAGTATATATATACATATATACAG
TGTATATATACTGTGTATATATACATGTATATATACTGTGTATATATACATGTATATATACTGTGTATAT
ATACATGTATATATACTGTGTATATATACATGTATATATATGTATACTGTATATATACTGTATATATAT
ATACACATATATACAGTATATATATACAGTATATACTGTATATATACAGTATATACGTGTATATATACA
TATATACAGTATATATGTAAATATACATATATACAGTATATATGTAAATATACATATATACATGTATAT
ATATACACTATATATATACATATATAGTGTATATATACATATATACATGTATATATTTACTATATGATT
CCATTTATATAAAGTGCCAAAACAGTCAAAAATAATCTATGTGGAAAAAATCAACAAAGGGATCCCC
CGGGCTGCAGGAATTCGATGGCGCGCCCTTTGGGGAGTCCTAAGAGGGCAGCTGGCAATGGACACCT
AGCAGTCCCTTTGAGACTTATTTCAGATGGAGCTGTAGAAAGATGCCATGGCTCACAGTGCCTCCCTG
GGAAGGGGGCAGAGGGCTGCCCAGTGAGGCCTCTTGCGAGCAGGAAATCACCAGAGACAAGGAAAG
ACCAGACCCCAGGATGACCTCAGTTAGGCCTTGCCCGACTGTCCTCAGAGTCCCATTCTCTGTGTCCTG
GTTCTTTTAGAAGATCATGGACCTCCAGGTCATTTCGTAACCGGAATCTGCCTGCGGGGGGTTTTGACA
AGCTATGGTATAGTGTATGTGGGGGTACTGACGAATTGGAAGATCATGGAGACCCCTTCTCCTCCTCC
ATCATTGGTCTGCCACATCCCTCCCAGGCGACTCACAGCAGAGAGACCTTGGATGTATGTAGGGTGCT
TTAAAACTCCAGCTGAGTTACAGTCTCTCCTTTCTGTTTTCACCTTAACCTTCCAGGGATGCAAACCCA
CGACAGGTTTAGCAGCAGAGTGGAGGCTGGCCATGAATCTCAGAGAAAGTGCTCACTGGAAAGGCTG
GTTTAGCCCAGGCCTGATGTGGAGGCACTGAGCTGGACGTTCTAGCGGGGTTGACACCCAACAGTTTA
CATAGGGGGAGGCCACCCCTCCTGAGCAGTCTCGGTGACTTGAAGAGGAAGCCGCTTCTTCTGTACCA
ACACAGAAGCTCCAGCGAACCCCCAGAATGCTGGCAGTGTGGGTGCTATGTAAAAGTATTTACATAGC
TTTGTAGAGTGAGCCAAGCCCAGTCTGTTTGGGATGACTCTTCACAGTGCCTCGAATCTGTCACACGTC
TTAGTAAGCAGAGTCACAGAGTTTCTGTCACATCATCCTCCTGCCTACAGGGAAGTAGGCCATGTCCC
TGCCCCCTACTCTGAGCCCAGCTGTGGGAGCCAGCCCTGCCCAATGGGCTCTCTCTGATTGGCTTCTCA
CTCACTTCTAAACTCCAGTGAGCAACTTCTCTCGGCTCGTTCAATTGGCGTGAAGGTCTGTGTCTTGCA
GAGAAGGTTCTTCACAACTGGGATAAAGGTCTCGCTGCTCAAGTGTAGCCCAGTAGAACTGCCAAGCC
CCTTCCCCTCCTCTCCCTAGACTCTTGGATGCAAGAAGAATCCAGGCAGCTCCAAGGGTGATTGTGTCC
AACCTAGAATGTCTTGAAAAAGACATTAAGGGGACTAGAGAAGACAGGGGATCCAACGGTTCTCTGC
AGCCCAGCCTGACTGACATGTAACTCTTCTGGTTCTCACCAGCCAGCTGGACCTGCTTAGTATTCTTTC
TGCCTCAGTTTCCCAGCCTGTACCCAGGGCTGTCATAGTTCCATTTCAGGCAGTAGTAATGAATGAGCT
GACATAAAACATTTAGAGCAGGGGTCAGTATGTATATAGAGTGATTATTCTATATCAGGCATTGCCTC
CTCGGAATGAAGCTTACAATCACCCCTCCCTCTGCAGTTCATCTTGGGGTGGCCAGAGGATCCAGCAG
ACACCTAGTGGGGTAACACACCCCAGCCAACTCGGCTGTTGCAGACTTTGTCTAGAAGTTTCACGTCT
CAGAGCTGAATTCCCTTCTCATGACCTTTGGCCGTGGGAGTGACACCTCACAGCTGTGGTGTTTTGACA
ACCAGCAGCCACTGGCACACAAAATGTGCAGCCAGCAGCATATGAAGTCCAAGAGGCGTCCCGGCCA
GCCCTGTCCTTGACCCCCACCTGACAATTAAGGCAAGAGCCTATAGTTTGCATCAGCAACAGTCACGG
TCAAAGTTTAGTCAATCAAACGTTGTGTAAGGACTCAACTATGGCTGACACGGGGGCCTGAGGCCTCC
CAACATTCATTAACAACAGCAAGTTCAATCATTATCTCCCCAAAGTTTATTGTGTTAGGTCAGTTCCAA
ACCGTGCTGACCATGGCTATGATCCAAAGGCCGGCCCCTTACGTCAGAGGCGAGCCTCCAGGTCCAGC
TGAGGGGCAGGGCTGTCCTCCCTTCTGTATACTATTTAAAGCGAGGAGGGCTAGCTACCAAGCACGGT
TGGCCTTCCCTCTGGGAACACACCCTTGGCCAACAGGGGAAATCCGGCGAGACGCTCTGAGATCCTGC
GAGAAGGAGGTGCGTCCTGCTGCCTGCCCCGGCACTCTGGCTCCCCAGCTCAAGGTTCAGGCCTTGCC
CCAGGCCGGGCCTCTGGGTACCTGAGGTCTTCTCCCGCTCTGTGCCCTTCTCATGGCGTCACGCATAGG
GTTGCGCATGCAGCTCATGCGGGAGCAGGCGCAGCAGGAGGAGCAGCGGGAGCGCATGCAGCAACA
GGCTGTCATGCATTACATGCAGCAGCAGCAGCAGCAGCAACAGCAGCAGCTCGGAGGGCCGCCCACC
CCGGCCATCAATACCCCCGTCCACTTCCAGTCGCCACCACCTGTGCCTGGGGAGGTGTTGAAGGTGCA
GTCCTACCTGGAGAATCCCACATCCTACCATCTGCAGCAGTCGCAGCATCAGAAGGTGCGGGAGTACC
TGTCCGAGACCTATGGGAACAAGTTTGCTGCCCACATCAGCCCAGCCCAGGGCTCTCCGAAACCCCCA
CCAGCCGCCTCCCCAGGGGTGCGAGCTGGACACGTGCTGTCCTCCTCCGCTGGCAACAGTGCTCCCAA
TAGCCCCATGGCCATGCTGCACATTGGCTCCAACCCTGAGAGGGAGTTGGATGATGTCATTGACAACA
TTATGCGTCTGGACGATGTCCTTGGCTACATCAATCCTGAAATGCAGATGCCCAACACGCTACCCCTGT
CCAGCAGCCACCTGAATGTGTACAGCAGCGACCCCCAGGTCACAGCCTCCCTGGTGGGCGTCACCAGC
AGCTCCTGCCCTGCGGACCTGACCCAGAAGCGAGAGCTCACAGATGCTGAGAGCAGGGCCCTGGCCA
AGGAGCGGCAGAAGAAAGACAATCACAACTTAATTGAAAGGAGACGAAGGTTCAACATCAATGACCG
CATCAAGGAGTTGGGAATGCTGATCCCCAAGGCCAATGACCTGGACGTGCGCTGGAACAAGGGCACC
ATCCTCAAGGCCTCTGTGGATTACATCCGGAGGATGCAGAAGGACCTGCAAAAGTCCAGGGAGCTGG
AGAACCACTCTCGCCGCCTGGAGATGACCAACAAGCAGCTCTGGCTCCGTATCCAGGAGCTGGAGATG
CAGGCTCGAGTGCACGGCCTCCCTACCACCTCCCCGTCCGGCATGAACATGGCTGAGCTGGCCCAGCA
GGTGGTGAAGCAGGAGCTGCCTAGCGAAGAGGGCCCAGGGGAGGCCCTGATGCTGGGGGCTGAGGTC
CCTGACCCTGAGCCACTGCCAGCTCTGCCCCCGCAAGCCCCGCTGCCCCTGCCCACCCAGCCACCATC
CCCATTCCATCACCTGGACTTCAGCCACAGCCTGAGCTTTGGGGGCAGGGAGGACGAGGGTCCCCCGG
GCTACCCCGAACCCCTGGCGCCGGGGCATGGCTCCCCATTCCCCAGCCTGTCCAAGAAGGATCTGGAC
CTCATGCTCCTGGACGACTCACTGCTACCGCTGGCCTCTGATCCACTTCTGTCCACCATGTCCCCCGAG
GCCTCCAAGGCCAGCAGCCGCCGGAGCAGCTTCAGCATGGAGGAGGGCGATGTGCTGTGAGAATTCC
GCGAATCAACCTCTGGATTACAAAATTTCTGAAAGATTGACTGGTATTCTTAACTATGTTGCTCCTTTT
ACGCTATGTGGATACGCTGCTTTAATGCCTTTGTATCATGCTATTGCTTCCCGTATGGCTTTCATTTTCT
CCTCCTTGTATAAATCCTGGTTGCTGTCTCTTTATGAGGAGTTGTGGCCCGTTGTCAGGCAACGTGGCG
TGGTGTGCACTGTGTTTGCTGACGCAACCCCCACTGGTTGGGGCATTGCCACCACCTGTCAACTCCTTT
CCGGGACTTACGCTTTCCCCCTCCCTATTGCCACGGCGGAACTCATCGCCGCCTGCCTTGCCCGCTGCT
GGACAGGGGCTCGGCTGTTGGGCACTGACAATTCCGTGGTGTTGTCGGGGAAGCTGACGTCCTTTCCA
TGGCTGCTCGCCTGTGTTGCCACCTGGATTCTGCGCGGGACGTCCTTCTGCTACGTCCCTTCGGCCCTC
AATCCAGCGGACCTTCCTTCCCGCGGCCTGCTGCCGGCTCTGCGGCCTCTTCCGCGTCTTCGCCTTCGC
CCTCAGACGACTCGGATCTCCCTTTGGGCCGCCTCCCCGCGGCGCGCCGACGCGCATGCTCCTCTAGA
CTCGAGGAATTCGGTACCCCGGGTTCGAAATCGATAAGCTTGATATCGAATTCCTGCAGGCTCAGAGG
CACACAGGAGTTTCTGGGCTCACCCTGCCCCCTTCCAACCCCTCAGTTCCCATCCTCCAGCAGCTGTTT
GTGTGCTGCCTCTGAAGTCCACACTGAACAAACTTCAGCCTACTCATGTCCCTAAAATGGGCAAACAT
TGCAAGCAGCAAACAGCAAACACACAGCCCTCCCTGCCTGCTGACCTTGGAGCTGGGGCAGAGGTCA
GAGACCTCTCTGGGCCCATGCCACCTCCAACATCCACTCGACCCCTTGGAATTTCGGTGGAGAGGAGC
AGAGGTTGTCCTGGCGTGGTTTAGGTAGTGTGAGAGGGTCCGGGTTCAAAACCACTTGCTGGGTGGGG
AGTCGTCAGTAAGTGGCTATGCCCCGACCCCGAAGCCTGTTTCCCCATCTGTACAATGGAAATGATAA
AGACGCCCATCTGATAGGGTTTTTGTGGCAAATAAACATTTGGTTTTTTTGTTTTGTTTTGTTTTGTTTTT
TGAGATGGAGGTTTGCTCTGTCGCCCAGGCTGGAGTGCAGTGACACAATCTCATCTCACCACAACCTT
CCCCTGCCTCAGCCTCCCAAGTAGCTGGGATTACAAGCATGTGCCACCACACCTGGCTAATTTTCTATT
TTTAGTAGAGACGGGTTTCTCCATGTTGGTCAGCCTCAGCCTCCCAAGTAACTGGGATTACAGGCCTGT
GCCACCACACCCGGCTAATTTTTTCTATTTTTGACAGGGACGGGGTTTCACCATGTTGGTCAGGCTGGT
CTAGAACTCCTGACCTCAAATGATCCACCCACCTAGGCCTCCCAAAGTGCACAGATTACAGGCGTGGG
CCACCGCACCTGGCCAAATTTTTAATTTTTTTCTAGAGATAGGGTCTTACTGTGTTGCCCAGGCTGGTG
TCAAACTCCTGGGCTCAAGCAGATCCTCCTGCCTCAGCTTCCCAAAGTGGTGGGATTATAGGTGTGAG
CCACTGCGCCCAGTCAGTAGCCCCCTCTTTGCCCCTCACTGAGCCCTACTGGATGTTCTTGGTTGTGTG
ACAGTTTCCCCATCTATTAAACAGAAACCCCTATAGCAGAGGGGAGGATGAGGTTGGAAAATCAGGA
GCATTGTTATTCTATTCTTGTGGGATCGGGGAAGCAGACATCTGGGTGGATGTTTGGGGAATGCTGGG
CTCAGTTGAGGAAGTAGGGGGGCCCCTGGGGCTTACAGGGACTGGAAGCTCTGAGCTGGCCAGAGGG
ATGTTGCAATCCTGCCAGGGTCTTGTCTATGCTGTCCCTTTCACAACCATCCCCCTACCGCCAGGCTGA
CACGTGGTTGTGGGGGCACAAGGCCAGCCGAACTAGAGTCTGAGGCTGGGCTGAGGACACCCTCCCC
ATCAGCTGCCAGGGTCACTGGCGGTCAAAGGCAGCTGGTGGGGAAGGAATTGGACTCCAGCCCTGGG
GGACGGATGTGGTGATGGTGGGAAGCAGGCTTGGTGCCAGGAGGGGCATCAGAGGGTGAATAAGAGC
AGATAGAGTGTTTGGGGGAGGTAGCCAGCCAAAGGGGGTGAGGCCCGGTGGAAGGGAAGAAGGGGC
ATACACTCAGAGCTTTGCAGCTGAAGGTTTTAATTTTTTGAGATGGGGTCTCACTCTGTCTCACCAGGC
TGGAGTGCAGTGGCGCAATCACAGCTCACTGCAGCCCGGGGGATCCGGAGAGCTCGTCGACGGCGCG
CCAATTCCTGCAGCCCGGGTGGCATCCCTGTGACCCCTCCCCAGTGCCTCTCCTGGCCCTGGAAGTTGC
CACTCCAGTGCCCACCAGCCTTGTCCTAATAAAATTAAGTTGCATCATTTTGTCTGACTAGGTGTCCTT
CTATAATATTATGGGGTGGAGGGGGGTGGTATGGAGCAAGGGGCAAGTTGGGAAGACAACCTGTAGG
GCCTGCGGGGTCTATTGGGAACCAAGCTGGAGTGCAGTGGCACAATCTTGGCTCACTGCAATCTCCGC
CTCCTGGGTTCAAGCGATTCTCCTGCCTCAGCCTCCCGAGTTGTTGGGATTCCAGGCATGCATGACCAG
GCTCAGCTAATTTTTGTTTTTTTGGTAGAGACGGGGTTTCACCATATTGGCCAGGCTGGTCTCCAACTC
CTAATCTCAGGTGATCTACCCACCTTGGCCTCCCAAATTGCTGGGATTACAGGCGTGAACCACTGCTCC
CTTCCCTGTCCTTCTGATTTTAAAATAACTATACCAGCAGGAGGACGTCCAGACACAGCATAGGCTAC
CTGGCCATGCCCAACCGGTGGGACATTTGAGTTGCTTGCTTGGCACTGTCCTCTCATGCGTTGGGTCCA
CTCAGTAGATGCCTGGCGCGCCTACCAGTAAAAAAGAAAACCTATTAAAAAAACACCACTCGACACG
GCACCAGCTCAATCAGTCACAGTGTAAAAAAGGGCCAAGTGCAGAGCGAGTATATATAGGACTAAAA
AATGACGTAACGGTTAAAGTCCACAAAAAACACCCAGAAAACCGCACGCGAACCTACGCCCAGAAAC
GAAAGCCAAAAAACCCACAACTTCCTCAAATCGTCACTTCCGTTTTCCCACGTTACGTCACTTCCCATT
TTAAGAAAACTACAATTCCCAACACATACAAGTTACTCCGCCCTAAAACCTACGTCACCCGCCCCGTT
CCCACGCCCCGCGCCACGTCACAAACTCCACCCCCTCATTATCATATTGGCTTCAATCCAAAATAAGGT
ATATTATTGATGATGTTT.
Mature ATT protein
(SEQ ID No. 9)
EDPQGDAAQKTDTSHHDQDHPTFNKITPNLAEFAFSLYRQLAHQSNSTNIFFSPVSIATAFAML
SLGTKADTHDEILEGLNFNLTEIPEAQIHEGFQELLRTLNQPDSQLQLTTGNGLFLSEGLKLVD
KFLEDVKKLYHSEAFTVNFGDTEEAKKQINDYVEKGTQGKIVDLVKELDRDTVFALVNYIFFKG
KWERPFEVKDTEEEDFHVDQVTTVKVPMMKRLGMFNIQHCKKLSSWVLLMKYLGNATAIFFLPD
EGKLQHLENELTHDIITKFLENEDRRSASLHLPKLSITGTYDLKSVLGQLGITKVFSNGADLSG
VTEEAPLKLSKAVHKAVLTIDEKGTEAAGAMFLEAIPMSIPPEVKFNKPFVFLMIEQNTKSPLF
MGKVVNPTQK

The highlighted amino acids are the positions mutated/deleted in the mutated protein ATZ.

### Materials and Methods

Cell Culture Studies.

The cDNA for human AAT was inserted in the pcDNA3.1 plasmid (Invitrogen). The pcDNA3.1-ATZ was generated by site-directed mutagenesis of the pcDNA3.1-AAT. Mouse hepatoma Hepa 1-6 cells (ATCC Catalog No. CRL-1830™) stably transfected with human ATZ (clone ATZ13) were cultured in DMEM with 10% fetal bovine serum (FBS), 5% penicillin/streptomycin, and 1 mg/ml of G418. ATZ13 cells were incubated in Met/Cys-free medium for 1 h at 37° C. followed by pulse labeling with 150 μCi/ml of Easy Tag Express Protein Labeling Mix (Perkin Elmer) in pulse medium for 30 min at 37° C. Cells were then rinsed with DMEM 5% FBS with Met and Cys (chase medium) and chased for different time points. Cells were lysed in Lysis Buffer (50 mM Tris-HCl pH7.4, 200 mM NaCl, 1% Triton X-100, 1 mM EDTA, 50 mM Hepes, 1× protease inhibitor) for 1 h at 4° C. Cell lysates were rotated overnight at 4° C. with the polyclonal rabbit anti-human AAT (Dako, cod. A0012). After addition of protein A sepharose conjugated (Sigma), the samples were incubated for 2 h at 4° C. After four washes with lysis buffer, samples were resuspended in Laemmli Buffer 2× (24 mM Tris-HCl pH 6.8, 0.8% SDS, 4% glycerol, 2.5% β-mercaptoethanol, 0.004% bromophenol blue) and boiled for 5 min. Cell extracts were loaded onto a 10% SDS-PAGE gel.

TFEB-3xFLAG HeLa stable cell lines (HeLa-CF7)⁶ and HeLa untransfected cells were cultured in DMEM with 10% fetal bovine serum (FBS) and 5% penicillin/streptomycin and transiently transfected with pcDNA3.1-ATZ using Lipofectamine 2000 (Invitrogen). 24 hours after transfection, media were harvested for ELISA and cells were washed once with cold phosphate-buffered saline (PBS) and scraped with Ripa buffer (50 mM Tris-HCl pH 7.4, 150 mM NaCl, 1% Triton X-100, 1 mM EDTA pH 8.0, 0.1% SDS) containing complete protease inhibitor cocktail (Sigma). Samples were incubated for 20 min at 4° C. and centrifuged at 13,200 rpm for 10 min. The pellet was discarded and cell lysates were used for Western blot analysis. After transfer to PVDF membrane, blots were blocked with TBS-Tween 20 containing 5% non-fat milk for 1 hr at room temperature followed by incubation with rabbit-anti human AAT (Dako) overnight at 4° C. Donkey anti-rabbit IgG-HRP (GE Healthcare, cod. NA934) and ECL (Pierce) were used for detection of ATZ. Equal gel loading was confirmed with immunoblot for actin (Novus Biological, cod. NB600-501).

To detect human ATZ, ELISA was performed on five independent liver specimens from each mouse. Nunc Maxisorp plates were coated with Cappel Goat anti-human AAT (MP Biomedicals, cod. 55111), and then blocked in PBS-0.1% Tween20 containing 5% nonfat milk. 0.1-1 μg of total protein from liver samples were loaded into the wells. Serial dilutions of purified human AAT were loaded to build a standard curve. Rabbit-anti human AAT (Dako) was used as capturing antibody and goat anti-rabbit IgG-HRP (Dako, cod. PO448) as secondary antibody.

HDAd Vectors.

HDAd-TFEB and HDAd-AFP both bear a PEPCK-WL expression cassette^{11,30} driving the expression of baboon alpha-fetoprotein (AFP) or human TFEB, respectively. HDAd was produced in 116 cells with the helper virus AdNG163 as described elsewhere³⁰. Helper virus contamination levels were determined as described elsewhere and were found to be <0.05%³⁰. DNA analyses of HDAd genomic structure was confirmed as described elsewhere³⁰.

Mice and Injections.

The PiZ transgenic mice³¹ were maintained on a C57/BL6 background. Injections of HDAd-TFEB, HDAd-AFP, or saline were performed in the retrorbital plexus of 3-month-old PiZ mice. Blood samples were collected at baseline, 1 and 4 weeks post-injection by retrorbital bleeding. Mice were sacrificed at 4 weeks post-injection for harvesting of liver samples.

Analyses of Serum and Liver Samples.

Serum samples were analyzed by ELISA for ATZ detection. For ATZ immunoblot, ATZ ELISA and western blots, liver samples were snap frozen in liquid nitrogen and stored at −80° C. Liver specimens were fixed in 4% PFA for 12 h and stored in 70% EtOH until process and embedded into paraffin blocks and cut into 10 μm sections. The sections were rehydrated and treated with amylase solution 0.5% (a-amylase type VI-B, Sigma) for 20 minutes and then stained with PAS reagent according to manufacturer's instructions (Bio-Optica). For ATZ immunofluorescence, 6-um sections were rehydrated, blocked, incubated overnight at 4° C. with polyclonal rabbit anti-human AAT (Dako) and then with donkey anti-rabbit 488 (AlexaFluor, cod. A-21206) for one hour at room temperature.

For co-staining LAMP-1 and ATZ, 6-μm thick paraffin sections of livers were de-waxed by standard techniques, hydrated in PBS pH7.4 and permeabilized with PBS, 0.2% Triton. Heat Induced Epitope Retrieval (HIER) using citrate buffer method (pH 6.0) for LAMP-1 and Proteolytic Induced Epitope Retrieval (PIER) by Proteinase K for ATZ, were performed to retrieve the antigen sites. The sections were then covered for 30 min with 75 mM NH₄Cl/PBS to reduce quenching and incubated for 1 h at room temperature with blocking solution (3% BSA, 5% donkey serum, 20 mM MgCl₂, 0.3% Tween 20 in PBS pH 7.4). The primary antibodies used were: rat monoclonal LAMP-1 (1D4B) (Santa Cruz Biotechnology, cod. sc-19992) and polyclonal rabbit anti-human AAT (Dako). The incubation for LAMP-1 was carried out overnight at 4° C. whereas that for AAT was done for 1 h at room temperature. The secondary antibodies made in donkey, were: AlexaFluor-594 anti-rat (Invitrogen, cod. A-21209) for LAMP-1 and AlexaFluor-488 (Invitrogen, cod. A-21206) for AAT. Nuclei were counterstained with dapi (Invitrogen). Finally, the stained liver sections were mounted in mowiol, cover-slipped and examined under a Zeiss LSM 710 confocal laser-scanning microscope. At least 3 animals of each type were used for the experiments in triplicate and representative images are shown.

Liver specimens were homogenized in Ripa buffer (50 mM Tris-HCl pH 7.4, 150 mM NaCl, 1% Triton X-100, 1 mM EDTA pH 8.0, 0.1% SDS) and complete protease inhibitor cocktail (Sigma). Western blot and ELISA on hepatic extracts and serum were performed following the protocol used for detecting AAT in cell media reported above. For LC-3 western blotting, 20 μg of total protein were loaded on 12% SDS-PAGE. After transfer to PVDF membrane, the blots were blocked in TBS-Tween20 containing 1% BSA for 1 hr at RT, then rabbit-anti LC-3 (Novus Biological, cod NB100-2220) was applied overnight at 4° C. Donkey anti-rabbit IgG-HRP (GE Healthcare, cod. NA934) and ECL (Pierce) was used for detection of AT. Equal gel loading was confirmed with immunoblot for actin (Novus Biological, cod NB600-501).

Momomer-polymer analysis was performed according to previous method¹⁴. Sirius red staining was performed on 10 μm liver sections which were rehydrated and stained for one hour in picro-sirius red solution (0.1% Sirius red in saturated aqueous solution of picric acid). After two changes of acidified water (5 ml acetic acid glacial in 1 liter of water), the sections were dehydrate in three changes of 100% ethanol, cleared in xylene and mounted in a resinous medium. Hydroxyproline content was measured by a spectrophotometric assay as an assessment of liver collagen content as previously described³² and expressed as micrograms of hydroxyproline per microgram of liver.

For caspase-12 and PARP western blotting, 20 μg of total protein were loaded on 10% SDS-PAGE and after transfer to PVDF membrane, the blots were blocked in TBS-Tween20 containing 5% non-fat milk or 1% BSA for 1 hr at room temperature followed by overnight 4° C. incubation with rat-anti caspase-12 (Sigma, cod. C7611) or rabbit-anti PARP-1 (Alexis Biochemical, ALX-210-219-R100). Goat anti-rat IgG-HRP (GE Healthcare, cod. NA935) or donkey anti-rabbit IgG-HRP (GE Healthcare, cod. NA934) were used for detection.

Electron Microscopy (EM) Studies.

For routine EM analysis the small pieces of liver were excised from PiZ mice injected with either saline or control HDAd-AFP vector or HDAd-TFEB and fixed in 1% glutaraldehyde in 0.2 M HEPES buffer. Then small blocks of the liver tissue were post-fixed in uranyl acetate and in OsO₄. After dehydration through a graded series of ethanol, the tissue samples were cleared in propylene oxide, embedded in the Epoxy resin (Epon 812) and polymerized at 60° C. for 72 h. From each sample, thin sections were cut with a Leica EM UC6 ultramicrotome. For immuno-EM analysis of ATZ distribution in hepatocytes, small pieces of liver tissue were fixed in a mixture of 4% paraformaldehyde and 0.4% glutaraldehyde in 0.2 M PHEM buffer, infused with 2.3 M sucrose, frozen in liquid nitrogen and sectioned in Leica EM FC7 cryoultratome. Cryosections were incubated with antibodies against ATT and then with protein A conjugated with 10 nm gold particles. Both cryo and Epon-812 plastic sections were further investigated using a FEI Tecnai-12 (FEI, Einhoven, The Netherlands) electron microscope equipped with an Veletta CCD camera for digital image acquisition. Quantification of ATT gold labeling densities over the lysosome-like organelle in hepatocytes was performed using iTEM software (Olympus SYS, Germany) according the previously describe method³³. Briefly, morphometric grid with 50 nm mesh was placed over profiles of lysosome-like structures. “Touch count” module of the iTEM software was used to quantify (i) number of gold particles and (ii) number of grid nodes inside the lysosome profile. Gold density was expressed in arbitrary units (gold particles per node). The organelle was defined as “lysosome” on the basis of the round/oval shape and presence of intraluminal vesicles as well as disorganized electron-dense and membrane material in the lumen¹⁵.

Statistical Analyses.

Data are expressed as mean values±standard deviation. Statistical significance was computed using the Student's 2 tail t-test. A p<0.05 was considered statistically significant.

### Results

To investigate whether TFEB-mediated enhancement of lysosomal degradation pathways and autophagy ameliorates the liver phenotype of AAT deficiency, the authors transfected a mouse hepa-1,6 cell line stably expressing the human ATZ protein (ATZ13 cell line) with a plasmid that expresses TFEB under the control of the CMV promoter. The ATZ13 cells were subjected to a pulse-chase radiolabeling with ³⁵S-labeled Cys and Met and the resulting cell lysates were analyzed by immunoprecipitation followed by SDS-PAGE analysis. This experiment showed that newly synthesized intracellular ATZ decreased more rapidly in TFEB-transfected cells as compared to control untreated cells (FIG. 1A). HeLa cells stably overexpressing TFEB (HeLa-CF7 cell line)⁶ and control HeLa cells were transfected with the plasmid expressing ATZ and after 24 hours media and cells were harvested for detection by ELISA and western blot, respectively, of the ATZ protein, which was reduced in HeLa-CF7 cells compared to control cells (FIGS. 1B and 1C).

The authors next generated an HDAd vector that expresses the human TFEB cDNA under the control of a liver-specific promoter (phosphoenolpyruvate carboxykinase (PEPCK)-promoter) derived from rat and a liver-specific enhancer (Locus Control Region (LCR) from the apoE locus)¹⁰ derived from human (HDAd-TFEB; FIG. 7) to investigate the therapeutic potential of HDAd-TFEB vector in the PiZ mouse, a transgenic mouse that expresses the human ATZ gene under control of its endogenous regulatory regions and recapitulates the features of liver disease observed in humans, i.e. intrahepatocytic ATZ-containing globules, inflammation/regenerative activity, and fibrosis⁴.

Human liver-specific promoter, as human phosphoenolpyruvate carboxykinase (PEPCK)-promoter can be similarly used.

The authors injected 3-month old PiZ mice (at least n=5 for each group) intravenously with the HDAd-TFEB vector at the dose of 1×10¹³ vp/kg. Control mice were injected with either saline or with the same dose of 1×10¹³ vp/kg of HDAd vector that expresses the unrelated, non-immunogenic, non-toxic alpha-fetoprotein (AFP) reporter gene under the control of the same expression cassette and within the same vector backbone¹¹ as the HDAd-TFEB vector (HDAd-AFP). No changes in appearance, behavior, and body weight were noted in mice injected with HDAd-TFEB compared to the two groups of control mice up to the time of sacrifice at 4 weeks post-injection. The livers of animals injected with HDAd-TFEB showed a dramatic reduction of both ATZ accumulation and ATZ-containing globules by periodic acid-Schiff (PAS) staining and by immunofluorescence, respectively, compared to saline or HDAd-AFP injected mice (FIGS. 2A and B). Consistent with TFEB-mediated activation of lysosome biogenesis^{6,8}, high levels of LAMP-1 were observed in livers of HDAd-TFEB injected animals (FIG. 2C). Interestingly, a negative correlation was noted between ATZ and LAMP-1 immunostaining signals and the few areas positive for ATZ signals did not show an increase in LAMP-1 expression (FIG. 2C). The reduction of ATZ protein levels was confirmed by both western blot (FIG. 2D) and ELISA on hepatic protein extracts (FIG. 2E). The effect of HDAd-TFEB was specific, and HDAd-AFP had no effect on hepatic ATZ levels (FIGS. 2A, B, and D). Besides the increase in LAMP-1 (FIG. 2C), an increase in LC3 levels was observed in the livers of mice injected with HDAd-TFEB, compared to saline or HDAd-AFP injected mice (FIG. 2F and FIG. 8). Taken together, these results demonstrated that hepatic gene transfer of TFEB enhances autophagy and lysosome biogenesis in the liver and reduces accumulation of ATZ in the liver of PiZ mice.

ATZ serum levels were reduced in mice injected with HDAd-TFEB vector at 4 weeks post-injection compared to baseline levels of the same mice. Animals injected with either saline or HDAd-AFP vector showed serum ATZ levels, which were not statistically different from baseline levels (FIG. 3).

Monomeric ATZ molecules bind together forming long, polymeric chains that reside in the endoplasmic reticulum (ER) of the cells in a conformation with a very long half-life. In the present study, PiZ mice were injected at the age of 3 months, when a significant hepatic accumulation of ATZ is already established, as shown by PAS staining and ATZ immunostaining¹²⁻¹³. To determine the effect of HDAd-mediated gene transfer of TFEB on monomer and polymer ATZ pools, the authors analyzed liver samples from HDAd-TFEB injected mice and the corresponding controls using a previously published assay¹⁴. First, ATZ polymers were isolated from the monomers in liver lysates under non-denaturing conditions and then the separated polymer and monomer fractions were denatured and compared by quantitative immunoblot. The denaturation step reduces the polymers to monomers and the resulting bands can be compared at the same molecular weight¹⁴. A statistically significant decrease in both ATZ monomer and polymer was observed in HDAd-TFEB injected mouse livers compared to either saline or HDAd-AFP injected control mice (FIG. 3).

Electron microscopy (EM) analysis of thin sections from livers of animals injected with saline or control HDAd-AFP vector revealed numerous membrane bound inclusions in hepatocyte cytoplasm (FIG. 4A-D). These inclusions ranged from smaller (0.3-1 μm; see FIG. 4A) to larger sizes which were comparable to nuclei (up to 10 μm; see FIG. 4B), and exhibited membrane continuity with cisternae of rough endoplasmic reticulum (RER) decorated by ribosomes (FIG. 4C, D). These features suggested that such structures are the sites of newly-synthesized ATZ which accumulates and aggregates in the RER. Indeed, immunolabelling of ATZ in thin cryosections indicated a strong concentration of ATZ in the inclusions (FIG. 5A, B). In contrast, most of the hepatocytes in HDAd-TFEB injected animals lack the inclusions observed in control PiZ mice (FIG. 4E, F), with exception of few cells that still contained large ATZ aggregates (FIG. 4G). Notably, these remaining aggregates were frequently surrounded by double membrane (FIG. 4G, H), that indicates their transformation into an autophagic vacuole. Immuno-EM revealed ATZ to be diffusely distributed along the RER profiles in the hepatocytes of HDAd-TFEB injected mice (FIG. 5C). In addition, significant amounts of the protein were detected within several multi-vesicular body (MVB)-like structures (FIG. 5D, E), which correspond to “lysosomes” or “autolysosomes” based on their ultrastructural features (see online Methods). The elevated ATZ signal in MVB-like structures indicates the activation of ATZ degradation by the lysosomal pathway upon TFEB gene transfer. Indeed, gold particles in lysosome-like organelles were frequently associated with intraluminal vesicles that are actively involved in lysosome degradation¹⁵. Notably, similar MVB-like structures in control saline-treated animals exhibited little or no ATZ compared to HDAd-TFEB injected mice (FIG. 5F, G), as shown by morphometric quantitative analysis (FIG. 5H). Taken together these data, showed that TFEB hepatic expression enhances degradation of insoluble hepatic ATZ in autolysosomes.

Hepatic fibrosis is a key feature of the hepatic disease that characterizes AAT deficiency and is secondary to hepatocyte apoptosis. Therefore, the authors next investigated whether TFEB gene transfer reduced ATZ-induced liver fibrosis. Collagen deposition was determined by Sirius red staining and by measurement of hepatic hydroxyproline content. HDAd-TFEB injection resulted in a reduction of Sirius red staining (FIG. 6A) and of hydroxyproline content of approximately 44% in the livers of HD-TFEB injected mice compared to saline injected animals (FIG. 6B).

Caspase-12 is related to ER stress-induced apoptosis in ATZ expressing cells and livers¹⁶. Therefore, the authors next investigated whether the reduction in the ATZ load of HDAd-TFEB injected mice resulted in reduced activation of caspase-12. Western blot analysis showed that the ˜42 KDa cleavage product that corresponds to activated caspase-12 is significantly reduced in HDAd-TFEB injected mouse livers compared to control livers (FIG. 6C). The authors also observed a reduction in caspase-cleaved 89 KDa and 24 KDa fragments of poly(ADP-ribose) polymerase-1 (PARP-1), which are generated during the execution of apoptotic program (FIG. 6D).

In summary, TFEB hepatic gene transfer reduced detrimental activation of liver apoptosis and fibrosis which underlines the pathogenesis of neonatal hepatitis, cirrhosis and hepatocellular carcinoma in AAT deficiency¹⁷.

### Discussion

Gene therapy strategies for liver disease of AAT deficiency have been investigated so far to avoid liver transplantation and have been designed with the goal of downregulating endogenous hepatocyte ATZ levels¹⁸. Previous attempts to correct the liver phenotype of AAT deficiency have been focused on the use of short hairpin RNA (shRNA) to silence the mutant ATZ¹⁹⁻²⁰. However, the use of shRNA delivered by gene therapy vectors has raised concerns because severe toxicity and lethality have been reported in mice²¹. shRNA-mediated saturation of the exportin-5 pathway, which shuttles cellular micro-RNA (miRNA) from the nucleus to the cytoplasm, has been proposed as the mechanism responsible for this severe toxic response²¹. To overcome this problem, strategies based on viral vector-mediated transfer of miRNA sequences targeting the AAT gene have been developed²².

In the present study, the authors have investigated a novel strategy to correct the hepatic disease of AAT deficiency, based on clearance of ATZ accumulation mediated by TFEB gene transfer. The current view is that ATZ is degraded by both proteasomal and autophagic pathway. The proteasome is responsible for degrading the soluble forms of ATZ by means of ER-associated degradation while autophagy is involved in the disposal of the insoluble ATZ polymers and aggregates²³. Autophagy has been previously shown to be involved in ATZ degradation^{4,13,24-25}. In the present study, the authors showed that TFEB gene transfer in liver resulted in the reduction of polymeric ATZ accumulation upon increased autophagy in the liver, as indicated by increased LAMP-1 staining (FIG. 2C) and by immunogold EM that showed increased ATZ signals within autolysosomes (FIG. 5).

The results of the present study clearly indicate that HDAd vector-mediated TFEB hepatic expression resulted in reduction of ATZ aggregates (FIG. 2) and liver injury, as shown by decrease in hepatic apoptosis and fibrosis (FIG. 6). Liver injury in patients with AAT deficiency is a direct consequence of hepatic accumulation of polymerized ATZ and therefore, TFEB gene transfer has tremendous potential for the treatment of liver disease in patients with AAT deficiency.

Previous work demonstrated that overexpression of TFEB promotes reduction of glycosaminoglycans by lysosomal exocytosis in a mouse model of lysosomal storage disorder due to deficiency of lysosomal enzymes⁸. In the present study, the authors show that TFEB gene transfer results in clearance of mutant, toxic protein which accumulates in a cell compartment that is not the lysosome. Therefore, the authors' data indicate that TFEB gene transfer may be effective for treatment of a wide spectrum of human disorders due to accumulation of toxic proteins, including neurodegenerative disorders.

Although the results of the present study were obtained with the HDAd vector, similar outcomes are expected with TFEB gene transfer achieved by other gene therapy vectors, such as AAV that has recently generated encouraging results in humans²⁶. Ultimately, the choice of the vector for clinical liver gene therapy will be dictated by a careful evaluation of efficacy and safety profile between the available vectors.

Up to one month after the injection, the HDAd-TFEB mice appeared in general good health and were undistinguishable from the control groups. Further studies are needed to investigate the safety of hepatic long-term TFEB expression. Nevertheless, the results of the present study illustrate the great potential of TFEB gene transfer, or possibly of pharmacological approaches resulting in TFEB activation, for therapy of liver disease caused by hepatotoxic ATZ. TFEB colocalizes with master growth regulator mTOR complex 1 (mTORC1) on the lysosomal membrane and in the presence of nutrients TFEB phosphorylation by mTORC1 inhibits TFEB activity²⁷. Conversely, pharmacological inhibition of mTORC1, as well as starvation and lysosomal disruption, activates TFEB by promoting its nuclear translocation. Small molecules inducing TFEB nuclear translocation and activity have been identified and could be used for therapeutic applications²⁷. Therefore, besides gene transfer, pharmacological induction of TFEB or TFEB target gene activation can be exploited to promote clearance of ATZ.

Genetic or environmental modifiers predispose a subgroup of homozygotes for the classical form of AAT deficiency to develop liver disease and/or protect the remainder population from hepatic disease³. Moreover, ATZ protein appears to also act as a modifier gene that exacerbates other forms of liver disease²⁸⁻²⁹. The results of the authors' study suggest that genetically determined differences in the level of activity of the TFEB-autophagy-lysosome axis may play a role in favoring or protecting from the development of liver disease.

In conclusion, the results of this study show the efficacy of TFEB gene transfer for therapy of AAT deficiency liver disease by ATZ disposal through the autophagolysosome system. TFEB gene transfer might provide an innovative therapeutic strategy for treatment of hepatic damage caused by AAT deficiency, which is a common cause of liver injury.

### REFERENCES

1. Sveger, T. Acta Paediatr Scand 77, 847-851 (1988).
2. Eriksson, S., Carlson, J. & Velez, R. N Engl J Med 314, 736-739 (1986).
3. Piitulainen, E., Carlson, J., Ohlsson, K. & Sveger, T. Chest 128, 2076-2081 (2005).
4. Hidvegi, T., et al. Science 329, 229-232 (2010).
5. Kaushal, S., et al. Exp Biol Med (Maywood) 235, 700-709 (2010).
6. Sardiello, M., et al. Science 325, 473-477 (2009).
7. Settembre, C., et al. Science 332, 1429-1433 (2011).
8. Medina, D. L., et al. Dev Cell 21, 421-430 (2011).
9. Brunetti-Pierri, N. & Ng, P. Hum Mol Genet 20, R7-13 (2011).
10. Brunetti-Pierri, N., et al. Mol Ther 12, 99-106 (2005).
11. Brunetti-Pierri, N., et al. Hum Gene Ther 17, 391-404 (2006).
12. Lindblad, D., Blomenkamp, K. & Teckman, J. Hepatology 46, 1228-1235 (2007).
13. Teckman, J. H., et al., Am J Physiol Gastrointest Liver Physiol 283, G1156-1165 (2002).
14. An, J. K., et al. Hepatology 41, 160-167 (2005).
15. Saftig, P. & Klumperman, J. Nat Rev Mol Cell Biol 10, 623-635 (2009).
16. Hidvegi, T., et al., J Biol Chem 280, 39002-39015 (2005).
17. Rudnick, D. A. & Perlmutter, D. H. Hepatology 42, 514-521 (2005).
18. McLean, C., Greene, C. M. & McElvaney, N. G. Biologics 3, 63-75 (2009).
19. Duan, Y. Y., et al. Gastroenterology 127, 1222-1232 (2004).
20. Cruz, P. E., et al. Lab Invest 87, 893-902 (2007).
21. Grimm, D., et al. Nature 441, 537-541 (2006).
22. Mueller, C., et al. Mol Ther 20, 590-600 (2012).
23. Perlmutter, D. H. Cell Death Differ 16, 39-45 (2009).
24. Teckman, J. H., et al., Am J Physiol Gastrointest Liver Physiol 278, G39-48 (2000).
25. Kamimoto, T., et al. J Biol Chem 281, 4467-4476 (2006).
26. Nathwani, A. C., et al. N Engl J Med 365, 2357-2365 (2011).
27. Settembre, C., et al. EMBO J31, 1095-1108 (2012).
28. Graziadei, I. W., et al. Hepatology 28, 1058-1063 (1998).
29. Campbell, K. M., et al. J Pediatr Gastroenterol Nutr 44, 99-103 (2007).
30. Palmer, D. & Ng, P. Mol Ther 8, 846-852 (2003).
31. Carlson, J. A., et al. J Clin Invest 82, 26-36 (1988).
32. Reddy, G. K. & Enwemeka, C. S. Clin Biochem 29, 225-229 (1996).
33. Mironov, A. A., et al. J Cell Riot 155, 1225-1238 (2001).
34. Stoller, J. K. & Aboussouan, L. S. Am J Respir Crit Care Med 185(3), 246-259 (2012).
35. Auricchio et al. Hum. Mol. Genet. 10(26):3075-81 (2001).
36. Vannucci et al. New Microbiologica 36:1-22 (2013).
37. Stephen et al. J Virol.; 84:9987-9994 (2010).
38. Kim et al. Proc Natl Acad Sci USA 98: 13282-13287 (2001). 39. Morral et al. Proc Natl Acad Sci USA. 1999; 96:12816-12821) (1999).

1-20. (canceled)

21. A method of treating a pathological condition characterized by a deficiency of alpha-1-antitrypsin (AAT), comprising administering an effective amount of a vector comprising a TFEB coding sequence under the control of a promoter able to efficiently express said TFEB coding sequence to a patient in need thereof.

22. The method of claim 21, wherein the TFEB coding sequence is hTFEB consisting essentially of the sequence of SEQ ID No. 3.

23. The method of claim 21, wherein the vector is a viral vector.

24. The method according to claim 23, wherein the vector is selected from the group consisting of adenoviral vectors, lentiviral vectors, retroviral vectors, Adeno associated vectors (AAV) and naked plasmid DNA vectors.

25. The method according to claim 24, wherein the vector is selected from the group consisting of helper-dependent adenoviral vectors.

26. The method according to claim 21, wherein the deficiency of AAT is due to a mutation of the AAT gene.

27. The method according to claim 26 wherein the mutation of the AAT gene causes a substitution of glutamate into lysine at amino acid position 342 and/or a substitution of serine into phenylalanine at amino acid position 53 (S_{iiyama}) and/or a deletion of phenylalanine at amino acid position 52 (Mₘₐₗₜₒₙ) of the AAT protein (SEQ ID No. 9).

28. The method according to claim 21, wherein said AAT deficiency is characterized by an accumulation of a wild type and/or mutated AAT protein in a tissue.

29. The method according to claim 28, wherein the accumulation of the wild type and/or mutated AAT protein further comprises the formation of wild type and/or mutated AAT aggregates in the tissue.

30. The method according to claim 28, wherein the tissue is liver.

31. The method according to claim 21, wherein the vector comprises a liver specific promoter and, optionally, regulatory sequences.

32. The method according to claim 31, wherein the liver specific promoter is phosphoenolpyruvate carboxykinase (PEPCK) promoter consisting essentially of the sequence of SEQ ID No. 1.

33. The method according to claim 31, wherein the liver regulatory sequence is the liver specific enhancer Locus Control Region (LCR) from the apoE locus consisting essentially of the sequence of SEQ ID No. 6.

34. The method according to claim 31, wherein the vector comprises the nucleotide sequence of SEQ ID No. 8.

35. A method of treating a pathological condition characterized by a deficiency of alpha-1-antitrypsin (AAT) comprising administering an effective amount of a host cell transformed by a vector comprising a TFEB coding sequence under the control of a promoter able to efficiently express said TFEB coding sequence to a patient in need thereof.

36. A method of treating a pathological condition characterized by a deficiency of alpha-1-antitrypsin (AAT) comprising administering an effective amount of a viral particle containing a vector comprising a TFEB coding sequence under the control of a promoter able to efficiently express said TFEB coding sequence to a patient in need thereof.

37. A method of treating a pathological condition characterized by a deficiency of alpha-1-antitrypsin (AAT) comprising administering an effective amount of a pharmaceutical composition comprising a vector comprising a TFEB coding sequence under the control of a promoter able to efficiently express said TFEB coding sequence and excipients to a patient in need thereof.

38. A method of treating a pathological condition characterized by a deficiency of alpha-1-antitrypsin (AAT), comprising administering an effective amount of the TFEB protein, synthetic or biotechnological functional derivative thereof, peptide fragments thereof, chimeric molecules comprising the TFEB protein, synthetic or biotechnological functional derivative thereof.

39. The method of claim 38, wherein the TFEB protein consists essentially of the amino acid sequence of SEQ ID No. 4.
